(19) United States
(12) Patent Application Publication
(10) Pub. No.: US 2023/0286927 A1
(43) Pub. Date: Sep. 14, 2023

(54) 2-PHENYLBENZOTRIAZOL-5-AMINE DERIVATIVES FOR THE TREATMENT AND PROPHYLAXIS OF HEPATITIS B VIRUS (HBV) INFECTION

(71) Applicant: Hoffmann-La Roche Inc., Little Falls, NJ (US)

(72) Inventors: Xianfeng LIN, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/318,947

(22) Filed: May 17, 2023

Related U.S. Application Data
(63) Continuation of application No. PCT/EP2021/082236, filed on Nov. 19, 2021.

(30) Foreign Application Priority Data
Nov. 20, 2020 (WO) PCT/CN2020/130577

(51) Int. Cl. C07D 249/20; C07D 405/12; C07D 417/12

(52) U.S. Cl. CPC C07D 249/20; C07D 405/12; C07D 417/12

(57) ABSTRACT

The present invention provides novel compounds having the general formula:

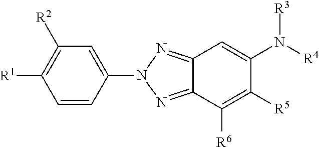

wherein R¹ to R⁶ are as described herein, compositions including the compounds and methods of using the compounds.

### CROSS-REFERENCE TO PRIOR APPLICATIONS

This application is a continuation of International Application No. PCT/EP2021/082236 having an International Filing Date of Nov. 19, 2021 and which claims benefit under 35 U.S.C. § 119 to International Application No. PCT/CN2020/130577 having an International Filing Date of Nov. 20, 2020. The entire contents of both are incorporated herein by reference.

### FIELD OF THE INVENTION

The present invention relates to organic compounds useful for therapy and/or prophylaxis of HBV infection in a mammal, and in particular to HBsAg (HBV Surface antigen) and HBeAg (HBV e antigen) inhibitors useful for treating HBV infection.

The present invention relates to 2-phenylbenzotriazol-5-amine derivatives of formula (I):

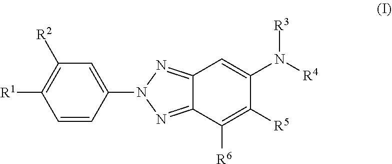

wherein R¹ to R⁶ are as described below, or a pharmaceutically acceptable salt, having pharmaceutical activity, their manufacture and pharmaceutical compositions containing them.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693;). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N. Engl. J. Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I)

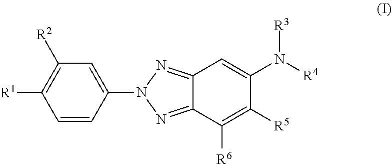

wherein
R¹ is H, halogen, C₁₋₆alkyl, C₁₋₆alkoxy, C₁₋₆alkylsulfonyl or C₁₋₆alkoxyC₁₋₆alkylsulfonyl;
R² is H, halogen, C₁₋₆alkyl, C₁₋₆alkoxy, C₁₋₆alkylsulfonyl or C₁₋₆alkoxyC₁₋₆alkylsulfonyl;
R³ is H, C₁₋₆alkyl, haloC₁₋₆alkyl, C₁₋₆alkoxyC₁₋₆alkyl, C₃₋₇cycloalkyl, C₃₋₇cycloalkylC₁₋₆alkyl, C₁₋₆alkylaminocarbonylC₁₋₆alkyl, heterocyclyl, heterocyclylC₁₋₆alkyl, C₁₋₆alkylheterocyclyl, (C₁₋₆alkyl)₂heterocyclyl, phenylheterocyclyl or —C(X)—R⁷; wherein
   X is O or S;
   R⁷ is amino, C₁₋₆alkyl, haloC₁₋₆alkyl, C₁₋₆alkylamino, (C₁₋₆alkyl)₂amino, C₃₋₇cycloalkyl, C₁₋₆alkylcarbonylamino, C₃₋₇cycloalkylcarbonylamino, phenylcarbonylamino or heterocyclyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy, ethoxy, and propoxy.

The term “C₃₋₇cycloalkyl” denotes to a saturated carbon mono or bicyclic ring or a saturated spiro-linked bicyclic carbon ring or a bridged carbon ring, containing from 3, 4, 5, 6, or 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇ cycloalkyl” group is cyclopropyl, cyclobutyl, cyclopentyl or cyclohexyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl, trifluoromethyl, trifluoromethyl, trifluoropropyl, or trifluoromethyletyl.

The term “heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Exemplary heterocyclyls are tetrahydrofuranyl, tetrahydrofuranyl, thiazolyl, 1,4-dioxanyl and tetrahydropyranyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, phenyl or heterocyclyl.

The term “carbonyl” alone or in combination refers to the group —C(O)—.

The term “sulfonyl” alone or in combination refers to the group —S(O)₂—.

The term “oxo” means an ═O group and may be attached to a carbon atom or a sulfur atom.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

### HBV Inhibitors

The present invention provides (i) a compound having the general formula (I):

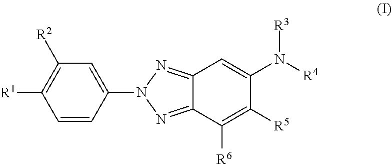

wherein
R¹ is H, halogen, C₁₋₆alkyl, C₁₋₆alkoxy, C₁₋₆alkylsulfonyl or C₁₋₆alkoxyC₁₋₆alkylsulfonyl;
R² is H, halogen, C₁₋₆alkyl, C₁₋₆alkoxy, C₁₋₆alkylsulfonyl or C₁₋₆alkoxyC₁₋₆alkylsulfonyl;
R³ is H, C₁₋₆alkyl, haloC₁₋₆alkyl, C₁₋₆alkoxyC₁₋₆alkyl, C₃₋₇cycloalkyl, C₃₋₇cycloalkylC₁₋₆alkyl, C₁₋₆alkylaminocarbonylC₁₋₆alkyl, heterocyclyl, heterocyclylC₁₋₆alkyl, C₁₋₆alkylheterocyclyl, (C₁₋₆alkyl)₂heterocyclyl, phenylheterocyclyl or —C(X)—R⁷; wherein
   X is O or S;
   R⁷ is amino, C₁₋₆alkyl, haloC₁₋₆alkyl, C₁₋₆alkylamino, (C₁₋₆alkyl)₂amino, C₃₋₇cycloalkyl, C₁₋₆alkylcarbonylamino, C₃₋₇cycloalkylcarbonylamino, phenylcarbonylamino or heterocyclyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

A further embodiment of the present invention is (ii) a compound of formula (I) according to (i), wherein
R¹ is H, halogen, C₁₋₆alkoxy, C₁₋₆alkylsulfonyl or C₁₋₆alkoxyC₁₋₆alkylsulfonyl;
R² is H, halogen or C₁₋₆alkoxy;
R³ is H, C₁₋₆alkyl, haloC₁₋₆alkyl, C₁₋₆alkoxyC₁₋₆alkyl, C₃₋₇cycloalkyl, C₃₋₇cycloalkylC₁₋₆alkyl, C₁₋₆alkylaminocarbonylC₁₋₆alkyl, tetrahydrofuranyl, tetrahydrofuranylC₁₋₆alkyl, C₁₋₆alkylthiazolyl, (C₁₋₆alkyl)₂thiazolyl, phenylthiazolyl or —C(X)—R⁷; wherein
   X is O or S;
   R⁷ is amino, C₁₋₆alkyl, haloC₁₋₆alkyl, C₁₋₆alkylamino, (C₁₋₆alkyl)₂amino, C₃₋₇cycloalkyl, C₁₋₆alkylcarbonylamino, C₃₋₇cycloalkylcarbonylamino, phenylcarbonylamino, tetrahydrofuranyl, tetrahydropyranyl or 1,4-dioxanyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

A further embodiment of the present invention is (iii) a compound of formula (I) according to (i), wherein
R¹ is H, F, Cl, methoxy, methylsulfonyl, ethylsulfonyl, isobutylsulfonyl or methoxyethylsulfonyl;
R² is H, F, Cl or methoxy;
R³ is H, methyl, ethyl, propyl, isopropyl, trifluoroethyl, trifluoromethyletyl, methoxyethyl, methoxypropyl, cyclobutyl, cyclopentyl, cyclopropylmethyl, cyclobutylmethyl, cyclopentylmethyl, methylaminocarbonylethyl, tetrahydrofuranyl, tetrahydrofuranylmethyl, methylthiazolyl, isopropylthiazolyl, (methyl)₂thiazolyl, phenylthiazolyl or —C(X)—R⁷;
   wherein
   X is O or S;
   R⁷ is amino, methyl, trifluoroethyl, trifluoropropyl, methylamino, dimethylamino, cyclopentyl, cyclohexyl, methylcarbonylamino, isopropylcarbonylamino, cyclopentylcarbonylamino, phenylcarbonylamino, tetrahydrofuranyl, tetrahydropyranyl or 1,4-dioxanyl;
R⁴ is H, methyl or ethyl;
R⁵ is H or methyl;
R⁶ is H or methyl;
wherein with the proviso that R¹, R² and R³ are not H simultaneously;
or a pharmaceutically acceptable salt thereof.

A further embodiment of the present invention is (iv) a compound of formula (I) according to (i), or a pharmaceutically acceptable salt thereof, wherein R¹ is H, halogen or C₁₋₆alkoxy.

A further embodiment of the present invention is (v) a compound of formula (I) according to (i), or a pharmaceutically acceptable salt thereof, wherein R¹ is H, F, Cl or methoxy.

A further embodiment of the present invention is (vi) a compound of formula (I) according to (i), or a pharmaceutically acceptable salt thereof, wherein
R³ is H, C₁₋₆alkyl, haloC₁₋₆alkyl, C₁₋₆alkoxyC₁₋₆alkyl, C₁₋₆alkylthiazolyl, (C₁₋₆alkyl)₂thiazolyl, phenylthiazolyl or —C(X)—R⁷; wherein
X is O or S;
R⁷ is amino, haloC₁₋₆alkyl, C₁₋₆alkylamino, (C₁₋₆alkyl)₂amino, C₁₋₆alkylcarbonylamino, phenylcarbonylamino or tetrahydropyranyl.

A further embodiment of the present invention is (vii) a compound of formula (I) according to (i), or a pharmaceutically acceptable salt thereof, wherein
R³ is H, ethyl, propyl, trifluoroethyl, methoxyethyl, methylthiazolyl, (methyl)₂thiazolyl, phenylthiazolyl or —C(X)—R⁷; wherein
   X is O or S;
   R⁷ is amino, trifluoropropyl, methylamino, dimethylamino, methylcarbonylamino, isopropylcarbonylamino, phenylcarbonylamino or tetrahydropyranyl.

A further embodiment of the present invention is (viii) a compound of formula (I) according to (i), or a pharmaceutically acceptable salt thereof, wherein
R¹ is H, halogen or C₁₋₆alkoxy;
R² is H, halogen or C₁₋₆alkoxy;
R³ is H, C₁₋₆alkyl, haloC₁₋₆alkyl, C₁₋₆alkoxyC₁₋₆alkyl, C₁₋₆alkylthiazolyl, (C₁₋₆alkyl)₂thiazolyl, phenylthiazolyl or —C(X)—R⁷; wherein
   X is O or S;
   R⁷ is amino, haloC₁₋₆alkyl, C₁₋₆alkylamino, (C₁₋₆alkyl)₂amino, C₁₋₆alkylcarbonylamino, phenylcarbonylamino or tetrahydropyranyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously.

A further embodiment of the present invention is (ix) a compound of formula (I) according to (i), or a pharmaceutically acceptable salt thereof, wherein
R¹ is H, F, Cl or methoxy;
R² is H, Cl or methoxy;
R³ is H, ethyl, propyl, trifluoroethyl, methoxyethyl, methylthiazolyl, (methyl)₂thiazolyl, phenylthiazolyl or —C(X)—R⁷; wherein
   X is O or S;
   R⁷ is amino, trifluoropropyl, methylamino, dimethylamino, methylcarbonylamino, isopropylcarbonylamino, phenylcarbonylamino or tetrahydropyranyl;
R⁴ is H, methyl or ethyl;
R⁵ is H or methyl;
R⁶ is H or methyl;
wherein with the proviso that R¹, R² and R³ are not H simultaneously.

In another embodiment (x) of the present invention, particular compounds of the present invention are selected from:
2-(3-chlorophenyl)benzotriazol-5-amine;
2-(4-chlorophenyl)benzotriazol-5-amine;
2-(4-methoxyphenyl)benzotriazol-5-amine;
2-(4-fluorophenyl)benzotriazol-5-amine;
2-(3-fluorophenyl)benzotriazol-5-amine;
2-(3-methoxyphenyl)benzotriazol-5-amine;
2-(4-methylsulfonylphenyl)benzotriazol-5-amine;
2-(4-ethylsulfonylphenyl)benzotriazol-5-amine;
2-[4-(2-methoxyethylsulfonyl)phenyl]benzotriazol-5-amine;
2-(4-isobutylsulfonylphenyl)benzotriazol-5-amine;
2-(4-chlorophenyl)-7-methyl-benzotriazol-5-amine;
2-(4-chlorophenyl)-N-methyl-benzotriazol-5-amine;
2-(4-chlorophenyl)-N-ethyl-benzotriazol-5-amine;
2-(4-chlorophenyl)-N-propyl-benzotriazol-5-amine;
2-(4-chlorophenyl)-N-(2-methoxyethyl)benzotriazol-5-amine;
N-cyclopentyl-2-phenyl-benzotriazol-5-amine;
N-(2-methoxyethyl)-2-(3-methoxyphenyl)benzotriazol-5-amine;
2-(3-chlorophenyl)-N-(2-methoxyethyl)benzotriazol-5-amine;
N-(2-methoxyethyl)-2-(4-methylsulfonylphenyl)benzotriazol-5-amine;
2-(4-fluorophenyl)-N-(tetrahydrofuran-3-ylmethyl)benzotriazol-5-amine;
2-(4-fluorophenyl)-N-tetrahydrofuran-3-yl-benzotriazol-5-amine;
2-(4-fluorophenyl)-N-(3-methoxypropyl)benzotriazol-5-amine;
2-(4-chlorophenyl)-N-(tetrahydrofuran-3-ylmethyl)benzotriazol-5-amine;
2-(4-chlorophenyl)-N-(cyclobutylmethyl)benzotriazol-5-amine;
2-(4-chlorophenyl)-N-(cyclopropylmethyl)benzotriazol-5-amine;
2-(4-chlorophenyl)-N-(cyclopentylmethyl)benzotriazol-5-amine;
2-(4-chlorophenyl)-N-(tetrahydrofuran-2-ylmethyl)benzotriazol-5-amine;
2-(4-chlorophenyl)-N-(2,2,2-trifluoroethyl)benzotriazol-5-amine;
2-(4-chlorophenyl)-N-cyclobutyl-benzotriazol-5-amine;
2-(4-chlorophenyl)-N-isopropyl-benzotriazol-5-amine;
2-(4-chlorophenyl)-N-cyclopentyl-benzotriazol-5-amine;
2-(4-chlorophenyl)-N-(2,2,2-trifluoro-1-methyl-ethyl)benzotriazol-5-amine;
2-(4-chlorophenyl)-N,N-dimethyl-benzotriazol-5-amine;
2-(4-chlorophenyl)-N,N-diethyl-benzotriazol-5-amine;
2-(4-chlorophenyl)-N-(2-methoxyethyl)-N-methyl-benzotriazol-5-amine;
2-(4-chlorophenyl)-N-ethyl-N-methyl-benzotriazol-5-amine;
3-[[2-(4-chlorophenyl)benzotriazol-5-yl]amino]-N-methyl-propanamide;
N-[2-(4-chlorophenyl)benzotriazol-5-yl]acetamide;
N-[2-(4-chlorophenyl)benzotriazol-5-yl]cyclohexanecarboxamide;
N-[2-(4-fluorophenyl)benzotriazol-5-yl]tetrahydrofuran-3-carboxamide;
N-[2-(4-fluorophenyl)benzotriazol-5-yl]tetrahydropyran-4-carboxamide;
N-[2-(4-fluorophenyl)benzotriazol-5-yl]tetrahydropyran-2-carboxamide;
N-[2-(4-fluorophenyl)benzotriazol-5-yl]cyclopentanecarboxamide;
3,3,3-trifluoro-N-[2-(4-fluorophenyl)benzotriazol-5-yl]propanamide;
N-[2-(4-chlorophenyl)benzotriazol-5-yl]tetrahydropyran-4-carboxamide;
N-[2-(4-chlorophenyl)benzotriazol-5-yl]tetrahydropyran-2-carboxamide;
N-[2-(4-chlorophenyl)benzotriazol-5-yl]-1,4-dioxane-2-carboxamide;
N-[2-(4-chlorophenyl)benzotriazol-5-yl]tetrahydrofuran-3-carboxamide;
N-[2-(4-chlorophenyl)benzotriazol-5-yl]cyclopentanecarboxamide;
N-[2-(4-chlorophenyl)benzotriazol-5-yl]tetrahydrofuran-2-carboxamide;
N-[2-(4-chlorophenyl)benzotriazol-5-yl]-3,3,3-trifluoro-propanamide;
N-[2-(4-chlorophenyl)benzotriazol-5-yl]-4,4,4-trifluoro-butanamide;
[2-(4-chlorophenyl)benzotriazol-5-yl]thiourea;
1-[2-(4-chlorophenyl)benzotriazol-5-yl]-3-methyl-thiourea;
N-[[2-(4-chlorophenyl)benzotriazol-5-yl]carbamothioyl]acetamide;
N-[[2-(4-chlorophenyl)benzotriazol-5-yl]carbamothioyl]cyclopentanecarboxamide;
N-[[2-(4-chlorophenyl)benzotriazol-5-yl]carbamothioyl]-2-methyl-propanamide;
N-[[2-(4-chlorophenyl)benzotriazol-5-yl]carbamothioyl]benzamide;
N-[[2-(4-chlorophenyl)-6-methyl-benzotriazol-5-yl]carbamothioyl]benzamide;
[2-(4-chlorophenyl)-6-methyl-benzotriazol-5-yl]thiourea;
3-[2-(4-chlorophenyl)benzotriazol-5-yl]-1,1-dimethyl-thiourea;
N-[2-(4-chlorophenyl)benzotriazol-5-yl]-4-isopropyl-thiazol-2-amine;
N-[2-(4-chlorophenyl)benzotriazol-5-yl]-4-phenyl-thiazol-2-amine;
N-[2-(4-chlorophenyl)benzotriazol-5-yl]-4-methyl-thiazol-2-amine; and
N-[2-(4-chlorophenyl)benzotriazol-5-yl]-4,5-dimethyl-thiazol-2-amine;
   or a pharmaceutically acceptable salt thereof.

In another embodiment (xi) of the present invention, particular compounds of the present invention are selected from:
2-(3-chlorophenyl)benzotriazol-5-amine;
2-(4-methoxyphenyl)benzotriazol-5-amine;
2-(3-methoxyphenyl)benzotriazol-5-amine;
2-(4-chlorophenyl)-7-methyl-benzotriazol-5-amine;
2-(4-chlorophenyl)-N-ethyl-benzotriazol-5-amine;
2-(4-chlorophenyl)-N-propyl-benzotriazol-5-amine;
2-(4-chlorophenyl)-N-(2,2,2-trifluoroethyl)benzotriazol-5-amine;
2-(4-chlorophenyl)-N,N-diethyl-benzotriazol-5-amine;
2-(4-chlorophenyl)-N-(2-methoxyethyl)-N-methyl-benzotriazol-5-amine;
N-[2-(4-fluorophenyl)benzotriazol-5-yl]tetrahydropyran-4-carboxamide;
N-[2-(4-chlorophenyl)benzotriazol-5-yl]tetrahydropyran-2-carboxamide;
N-[2-(4-chlorophenyl)benzotriazol-5-yl]-4,4,4-trifluoro-butanamide;
1-[2-(4-chlorophenyl)benzotriazol-5-yl]-3-methyl-thiourea;
N-[[2-(4-chlorophenyl)benzotriazol-5-yl]carbamothioyl]acetamide;
N-[[2-(4-chlorophenyl)benzotriazol-5-yl]carbamothioyl]-2-methyl-propanamide;
N-[[2-(4-chlorophenyl)benzotriazol-5-yl]carbamothioyl]benzamide;
N-[[2-(4-chlorophenyl)-6-methyl-benzotriazol-5-yl]carbamothioyl]benzamide;
[2-(4-chlorophenyl)-6-methyl-benzotriazol-5-yl]thiourea;
3-[2-(4-chlorophenyl)benzotriazol-5-yl]-1,1-dimethyl-thiourea;
N-[2-(4-chlorophenyl)benzotriazol-5-yl]-4-phenyl-thiazol-2-amine;
N-[2-(4-chlorophenyl)benzotriazol-5-yl]-4-methyl-thiazol-2-amine; and
N-[2-(4-chlorophenyl)benzotriazol-5-yl]-4,5-dimethyl-thiazol-2-amine;
   or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁷, are as defined above unless otherwise indicated. Furthermore, and unless explicitly otherwise stated, all reactions, reaction conditions, abbreviations and symbols have the meanings well known to a person of ordinary skill in organic chemistry.

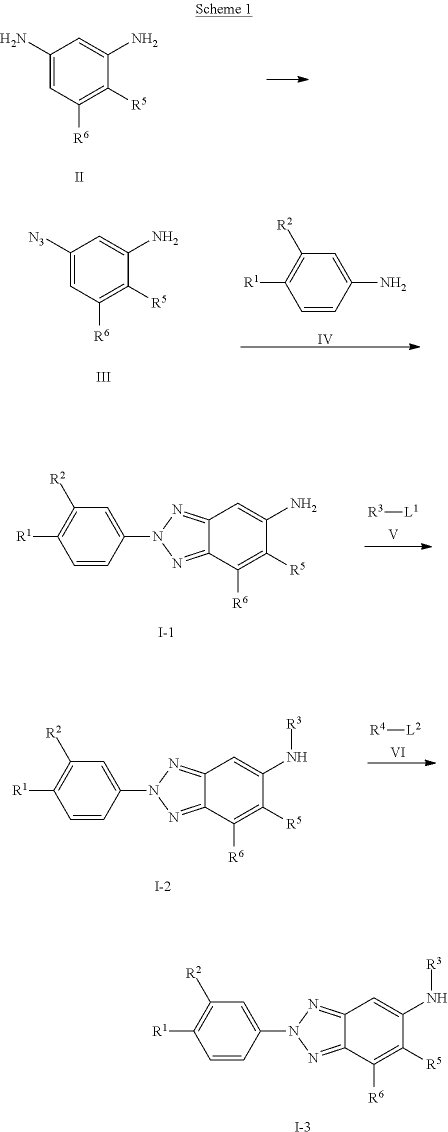

Wherein L¹ is N═C═S, halogen or OH; L² is halogen.

A compound of formula I can be prepared according to Scheme 1. The nitrosation of a primary aromatic amine II with nitrous acid, generated in situ from sodium nitrite and a strong acid, such as hydrochloric acid, sulfuric acid, or HBF₄, leads to a diazonium salt III, which was coupled with a compound of formula IV, followed by cyclization in the presence of copper (II) sulfate, ammonium hydroxide and a suitable solvent, such as pyridine, leads to a compound of formula I-1. The compound of formula I-2 can be prepared from coupling of the compound of formula I-1 with a compound of formula V in a suitable base, such as K₂CO₃ or Et₃N, in a suitable solvent, such as acetonitrile, THF or DCM. The compound of formula I-2 can also be prepared by coupling of a compound of formula I-1 with compound V in a suitable Lewis acid, such as p-toluenesulfonic acid in a suitable solvent, such as 1-butanol. The compound of formula I-3 can be prepared from coupling of a compound of formula I-2 with a compound of formula VI in a suitable base, such as K₂CO₃ or Et₃N, in a suitable solvent, such as acetonitrile, THF or DCM.

This invention also relates to a process for the preparation of a compound of formula (I) comprising at least one of the following steps:
(a) coupling and cyclization of a diazonium salt (III),

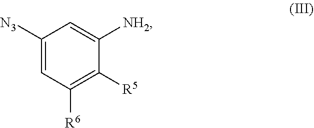

with a compound of formula (IV),

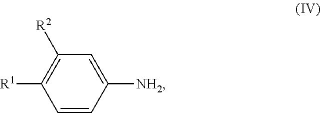

in the presence of copper (II) sulfate and ammonium hydroxide;
(b) coupling of a compound of formula (I-1),

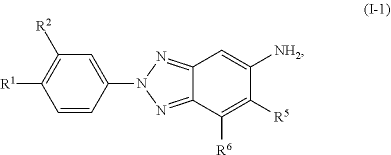

with a compound of formula (V), R³-L¹ (V), in the presence of a base or a Lewis acid;
(c) coupling of a compound of formula (1-2),

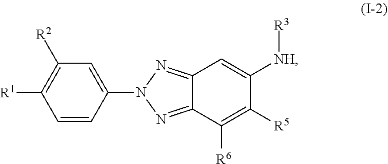

with a compound of formula (VI), R⁴-L² (VI), in the presence of a base;
wherein L¹ is N═C═S, halogen or OH; L² is halogen.
The base in step (b) can be for example K₂CO₃ or Et₃N;
The Lewis acid in step (b) can be for example p-toluenesulfonic acid;
The base in step (c) can be for example K₂CO₃ or Et₃N;

A compound of formula (I) when manufactured according to the above process is also an object of the invention.

The compound of this invention also shows good safety and PK profile.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

### Indications and Methods of Treatment

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

The invention relates in particular to a compound of formula (I) for use in the treatment or prophylaxis of HBV infection.

### EXAMPLES

The invention will be more fully understood by reference to the following examples. They should not, however, be construed as limiting the scope of the invention.

Abbreviations used herein are as follows:
ACN: acetonitrile
CDCl₃: deuterated chloroform
CD₃OD: deuterated methanol
DMF: dimethylformamide
DMSO-d₆: deuterated dimethylsulfoxide
EtOAc/EA: ethyl acetate
HPLC: high performance liquid chromatography
h: hour
IC₅₀: the half maximal inhibitory concentration
LC-MS: liquid chromatography-mass spectrometry
MeOH: methanol
m-CPBA: 3-chloroperoxybenzoic acid
MHz: megahertz
min: minute
mL: milliliter
mmol: millimole
MS (ESI): mass spectroscopy (electron spray ionization)
NMR: nuclear magnetic resonance
obsd. observed
PE petroleum ether
TEA: triethylamine
TFA: trifluoroacetic acid
THF: tetrahydrofuran
TLC: thin layer chromatography
δ: chemical shift
r.t.: room temperature

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using an Acquity Ultra Performance LC—3100 Mass Detector or Acquity Ultra Performance LC-SQ Detector. Standard LC/MS conditions were as follows (running time 3 minutes):
Acidic condition: A: 0.1% formic acid in H₂O; B: 0.1% formic acid in acetonitrile;
Basic condition: A: 0.05% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### Preparative Examples

The invention will be more fully understood by reference to the following examples. They should not, however, be construed as limiting the scope of the invention.

### Example 1

### 2-(3-Chlorophenyl)benzotriazol-5-amine

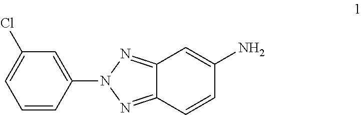 [SMILES: Nc1ccc2nn(-c3cccc(Cl)c3)nc2c1]

The title compound was prepared according to the following scheme:

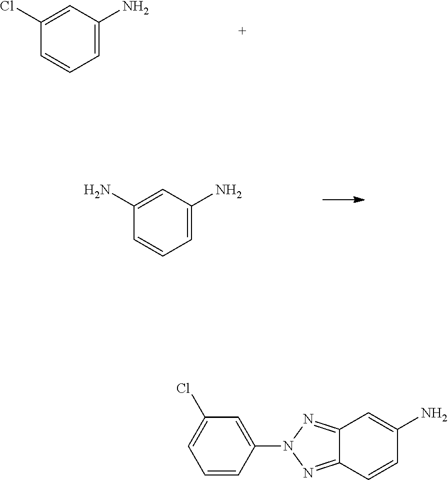 [SMILES: Nc1cccc(Cl)c1 ; Nc1cccc(N)c1 ; Nc1ccc2nn(-c3cccc(Cl)c3)nc2c1]

### Preparation of 2-(3-chlorophenyl)benzotriazol-5-amine

An aqueous solution (3.0 mL) of sodium nitrite (129.8 mg, 1.88 mmol, 1.2 eq) was added dropwise to a solution of m-phenylenediamine (169.5 mg, 1.57 mmol, 1 eq) in hydrochloric acid (10.0 mL) at 0° C. After 15 min, ammonium sulfamate (268.3 mg, 2.35 mmol, 1.5 eq) was added to the above mixture, then the mixture was stirred at 20° C. for 15 min. After adjusting the pH=5 using sodium acetate, 3-chlorobenzenamine (0.17 mL, 1.57 mmol, 1 eq) was added and the mixture was further stirred at 20° C. for 2 h. After the reaction was completed, the resulting mixture was basified to pH=9 with 1 mol/L NaOH solution, diluted with EA (50 mL), washed with brine (10 mL*2). The organic layer was dried over anhydrous sodium sulfat, and then concentrated to afford a red solid which was used in the next step without further purification.

A solution of copper (II) sulfate (1676.5 mg, 10.5 mmol, 6.7 eq) in ammonium hydroxide (12.0 mL) was added to the above red solid in pyridine (8 mL), then the reaction mixture was refluxed at 120° C. for 16 h. After the reaction was completed, the resulting mixture was diluted with EA (20 mL), washed with brine (5 mL*2). The organic layer was dried over anhydrous sodium sulfat, and concentrated. The crude product was purified by prep-HPLC (NH₃·H₂O) to afford 2-(3-chlorophenyl)benzotriazol-5-amine (110.5 mg, 0.45 mmol, 28.6% yield) as a light brown solid. MS obsd. (ESI⁺) [(M+H)⁺]: 245.0. ¹H NMR (400 MHz, DMSO-d₆) δ ppm: 8.22-8.12 (m, 2H), 7.71 (d, J=9.2 Hz, 1H), 7.66-7.59 (m, 1H), 7.56-7.49 (m, 1H), 7.02 (dd, J=1.9, 9.2 Hz, 1H), 6.69 (d, J=1.5 Hz, 1H), 5.74 (s, 2H).

### Example 2

### 2-(4-Chlorophenyl)benzotriazol-5-amine

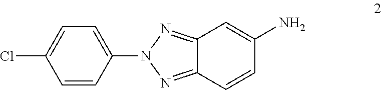 [SMILES: Nc1ccc2nn(-c3ccc(Cl)cc3)nc2c1]

The title compound was prepared in analogy to the procedure described for the preparation of Example 1, by using 4-chloroaniline instead of 3-chloroaniline. The product was purified by preparative HPLC to afford Example 2. MS obsd. (ESI⁺) [(M+H)⁺]: 245.1. ¹H NMR (400 MHz, CDCl₃) δ ppm: 8.17-8.14 (m, 2H), 7.63 (d, J=13.3 Hz, 1H), 7.41 (d, J=11.6 Hz, 2H), 6.86-6.83 (m, 2H), 3.90 (br, s, 2H).

### Example 3

### 2-(4-Methoxyphenyl)benzotriazol-5-amine

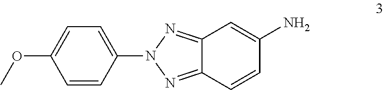 [SMILES: COc1ccc(-n2nc3ccc(N)cc3n2)cc1]

The title compound was prepared in analogy to the procedure described for the preparation of Example 1, by using 4-methoxyaniline instead of 3-chloroaniline. The product was purified by preparative HPLC to afford Example 3. MS obsd. (ESI⁺) [(M+H)⁺]: 241.1. ¹H NMR (400 MHz, DMSO-d₆) δ ppm: 8.23-8.04 (m, 2H), 7.74-7.60 (m, 1H), 7.21-7.09 (m, 2H), 6.96 (dd, J=1.9, 9.1 Hz, 1H), 6.70 (d, J=1.4 Hz, 1H), 5.59 (s, 2H).

### Example 4

### 2-(4-Fluorophenyl)benzotriazol-5-amine

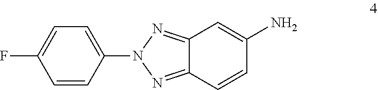 [SMILES: Nc1ccc2nn(-c3ccc(F)cc3)nc2c1]

The title compound was prepared in analogy to the procedure described for the preparation of Example 1, by using 4-fluoroaniline instead of 3-chloroaniline. The product was purified by preparative HPLC to afford Example 4. MS obsd. (ESI⁺) [(M+H)⁺]: 229.1. ¹H NMR (400 MHz, DMSO-d₆) δ ppm: 8.29-8.17 (m, 2H), 7.70 (d, J=9.2 Hz, 1H), 7.51-7.39 (m, 2H), 6.99 (dd, J=1.9, 9.1 Hz, 1H), 6.70 (d, J=1.4 Hz, 1H), 5.66 (s, 2H).

### Example 5

### 2-(3-Fluorophenyl)benzotriazol-5-amine

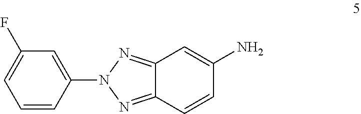 [SMILES: Nc1ccc2nn(-c3cccc(F)c3)nc2c1]

The title compound was prepared in analogy to the procedure described for the preparation of Example 1, by using 3-fluoroaniline instead of 3-chloroaniline. The product was purified by preparative HPLC to afford Example 5. MS obsd. (ESI⁺) [(M+H)⁺]: 229.1. ¹H NMR (400 MHz, DMSO-d₆) δ ppm: 8.04 (dd, J=1.2, 8.2 Hz, 1H), 7.96 (td, J=2.2, 10.2 Hz, 1H), 7.75-7.69 (m, 1H), 7.64 (dt, J=6.3, 8.3 Hz, 1H), 7.31 (dt, J=2.4, 8.4 Hz, 1H), 7.01 (dd, J=2.0, 9.2 Hz, 1H), 6.68 (d, J=1.5 Hz, 1H), 5.75 (br s, 2H).

### Example 6

### 2-(3-Methoxyphenyl)benzotriazol-5-amine

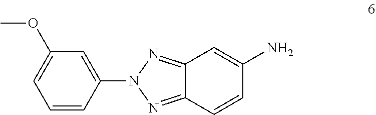 [SMILES: COc1cccc(-n2nc3ccc(N)cc3n2)c1]

The title compound was prepared in analogy to the procedure described for the preparation of Example 1, by using 3-methoxyaniline instead of 3-chloroaniline. The product was purified by preparative HPLC to afford Example 6. MS obsd. (ESI⁺) [(M+H)⁺]: 241.1. ¹H NMR (400 MHz, DMSO-d₆) δ ppm: 7.78 (td, J=1.1, 7.2 Hz, 1H), 7.74-7.67 (m, 2H), 7.49 (t, J=8.2 Hz, 1H), 7.07-6.93 (m, 2H), 6.69 (d, J=1.4 Hz, 1H), 5.68 (s, 2H), 3.88 (s, 3H).

### Example 7

### 2-(4-Methylsulfonylphenyl)benzotriazol-5-amine

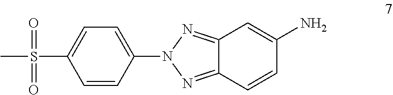 [SMILES: CS(=O)(=O)c1ccc(-n2nc3ccc(N)cc3n2)cc1]

The title compound was prepared in analogy to the procedure described for the preparation of Example 1, by using 4-methylsulfonylaniline instead of 3-chloroaniline. The product was purified by preparative HPLC to afford Example 7. MS obsd. (ESI⁺) [(M+H)⁺]: 288.0. ¹H NMR (400 MHz, DMSO-d₆) δ ppm: 8.42 (d, J=8.8 Hz, 2H), 8.13 (d, J=8.8 Hz, 2H), 7.73 (d, J=9.2 Hz, 1H), 7.04 (dd, J=1.9, 9.2 Hz, 1H), 6.67 (d, J=1.5 Hz, 1H), 5.82 (s, 2H), 3.29 (s, 3H).

### Example 8

### 2-(4-Ethylsulfonylphenyl)benzotriazol-5-amine

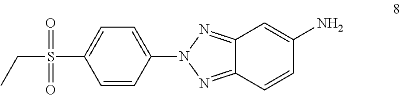 [SMILES: CCS(=O)(=O)c1ccc(-n2nc3ccc(N)cc3n2)cc1]

The title compound was prepared according to the following scheme:

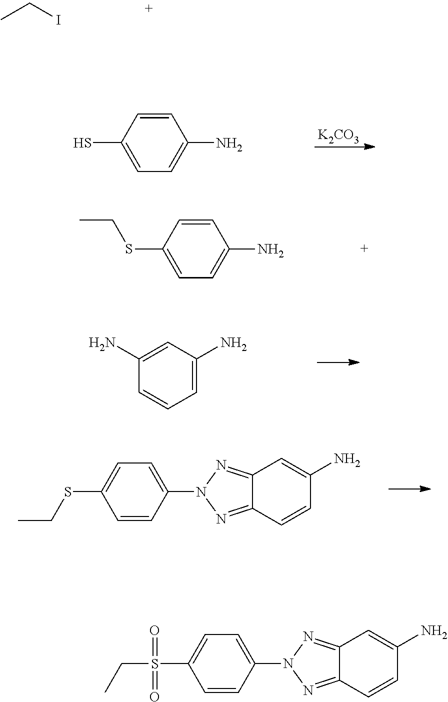

### Step 1: Preparation of 4-ethylsulfonylaniline

To a mixture of 4-aminothiophenol (2.0 g, 15.98 mmol, 1 eq) and potassium carbonate (4.42 g, 31.95 mmol, 2 eq) in anhydrous DMF (30 mL) solution was added iodoethane (1.41 mL, 17.57 mmol, 1.1 eq) slowly at 0° C. Then the mixture was stirred at 0° C. for 2 h. After the reaction was completed, the resulting mixture was diluted with water (30 mL), extracted with EA (50 mL*3), washed with brine and dried over Na₂SO₄. The combined organic phase was concentrated to afford crude 4-ethylsulfanylaniline (2.2 g, 14.36 mmol, 89.8% yield) as a brown oil which was used in the next step directly.

### Step 2: Preparation of 2-(4-ethylsulfanylphenyl)benzotriazol-5-amine

2-(4-Ethylsulfanylphenyl)benzotriazol-5-amine was prepared in analogy to the procedure described for the preparation of Example 1, by using 4-ethylsulfonylaniline instead of 3-chloroaniline. The crude product was used in the next step without further purification.

### Step 3: Preparation of 2-(4-ethylsulfonylphenyl)benzotriazol-5-amine

To a mixture of 2-(4-ethylsulfanylphenyl)benzotriazol-5-amine (150.0 mg, 0.55 mmol, 1 eq) in THF (5 mL) was added peracetic acid (1.5 mL). Then the mixture was stirred at 25° C. for 2 h. After the reaction was completed, the resulting mixture was quenched with aq. Na₂SO₃, basefied with aq. NaHCO₃, extracted with EA (20 mL*3), washed with brine and dried over Na₂SO₄. The combined organic phase was concentrated and purified by prep-HPLC (FA) to afford 2-(4-ethylsulfonylphenyl)benzotriazol-5-amine (15.1 mg, 0.05 mmol, 8.7% yield) as a light green solid. MS obsd. (ESI⁺) [(M+H)⁺]: 302.0. ¹H NMR (400 MHz, CDCl₃) δ ppm: 8.50 (d, J=8.8 Hz, 2H), 8.06 (d, J=8.8 Hz, 2H), 7.74 (d, J=9.0 Hz, 1H), 6.99-6.86 (m, 2H), 4.05 (br s, 2H), 3.18 (q, J=7.4 Hz, 2H), 1.33 (t, J=7.5 Hz, 3H).

### Example 9

### 2-[4-(2-Methoxyethylsulfonyl)phenyl]benzotriazol-5-amine

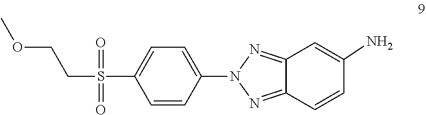 [SMILES: COCCS(=O)(=O)c1ccc(-n2nc3ccc(N)cc3n2)cc1]

The title compound was prepared in analogy to the procedure described for the preparation of Example 8, by using 1-bromo-2-methoxy-ethane instead of iodoethane. The product was purified by preparative HPLC to afford Example 9. MS obsd. (ESI⁺) [(M+H)⁺]: 332.0. ¹H NMR (400 MHz, CDCl₃) δ ppm: 8.52-8.45 (m, 2H), 8.11-8.03 (m, 2H), 7.74 (dd, J=0.8, 9.0 Hz, 1H), 7.01-6.89 (m, 2H), 4.05 (br s, 2H), 3.79 (t, J=6.1 Hz, 2H), 3.45 (t, J=6.1 Hz, 2H), 3.25 (s, 3H).

### Example 10

### 2-(4-Isobutylsulfonylphenyl)benzotriazol-5-amine

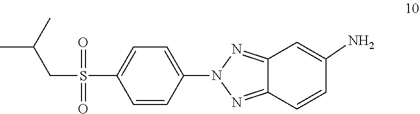 [SMILES: CC(C)CS(=O)(=O)c1ccc(-n2nc3ccc(N)cc3n2)cc1]

The title compound was prepared in analogy to the procedure described for the preparation of Example 8, by using 1-bromo-2-methyl-propane instead of iodoethane. The product was purified by preparative HPLC to afford Example 10. MS obsd. (ESI⁺) [(M+H)⁺]: 330.0. ¹H NMR (400 MHz, DMSO-d₆) δ ppm: 8.47-8.40 (m, 2H), 8.15-8.07 (m, 2H), 7.73 (d, J=9.2 Hz, 1H), 7.03 (dd, J=1.9, 9.2 Hz, 1H), 6.67 (d, J=1.4 Hz, 1H), 5.83 (s, 2H), 3.29 (d, J=6.4 Hz, 2H), 2.05 (quind, J=6.7, 13.3 Hz, 1H), 0.99 (d, J=6.7 Hz, 6H).

### Example 11

### 2-(4-Chlorophenyl)-7-methyl-benzotriazol-5-amine

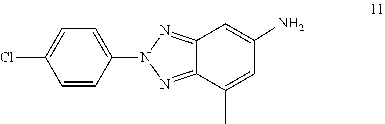 [SMILES: Cc1cc(N)cc2nn(-c3ccc(Cl)cc3)nc12]

The title compound was prepared in analogy to the procedure described for the preparation of Example 1, by using 4-chloroaniline instead of 3-chloroaniline & 5-methylbenzene-1,3-diamine instead of benzene-1,3-diamine. The product was purified by preparative HPLC to afford Example 11. MS obsd. (ESI⁺) [(M+H)⁺]: 259.1. ¹H NMR (400 MHz, CDCl₃) δ ppm: 8.23 (d, J=8.8 Hz, 2H), 7.48 (d, J=8.8 Hz, 2H), 6.75 (d, J=1.6 Hz, 1H), 6.68 (s, 1H), 3.90 (br, s, 2H), 2.63 (s, 3H).

### Example 12

### 2-(4-Chlorophenyl)-N-methyl-benzotriazol-5-amine

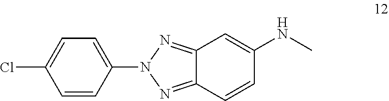 [SMILES: CNc1ccc2nn(-c3ccc(Cl)cc3)nc2c1]

The title compound was prepared according to the following scheme:

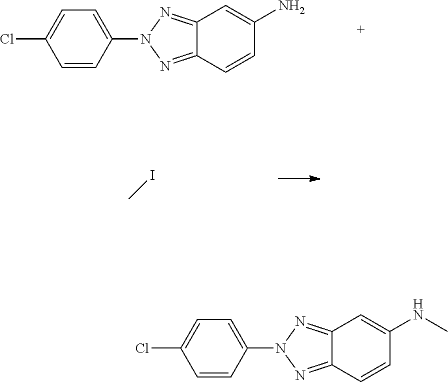

### Preparation of 2-(4-chlorophenyl)-N-methyl-benzotriazol-5-amine

K₂CO₃ (121 mg, 0.88 mmol, 2 eq) was added to the solution of iodomethane (233 mg, 1.64 mmol) and 2-(4-chlorophenyl)benzotriazol-5-amine (Example 2, 200 mg, 0.82 mmol, 1 eq) in acetonitrile (5 ml) at r.t. in a sealed tube. Then the mixture was stirred at 90° C. overnight. Then the mixture was filtered and concentrated. The resulting residue was purified by preparative HPLC to afford Example 12. MS obsd. (ESI⁺) [(M+H)⁺]: 259.1. ¹H NMR (400 MHz, DMSO-d₆) δ ppm: 8.20 (d, J=12.0 Hz, 2H), 7.71-7.65 (m, 3H), 7.02 (d, J=11.6 Hz, 2H), 6.46 (s, 1H), 6.37 (d, J=4.8 Hz, 1H), 2.77 (d, J=4.8 Hz, 3H).

### Example 13

### 2-(4-Chlorophenyl)-N-ethyl-benzotriazol-5-amine

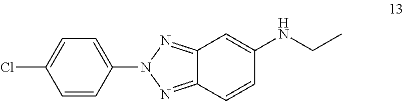 [SMILES: CCNc1ccc2nn(-c3ccc(Cl)cc3)nc2c1]

The title compound was prepared in analogy to the procedure described for the preparation of Example 12, by using bromoethane instead of iodomethane. The product was purified by preparative HPLC to afford Example 13. MS obsd. (ESI⁺) [(M+H)⁺]: 273.0. ¹H NMR (400 MHz, DMSO-d₆) δ ppm: 8.24-8.14 (m, 2H), 7.74-7.60 (m, 3H), 7.04 (dd, J=2.0, 9.2 Hz, 1H), 6.49 (d, J=1.5 Hz, 1H), 6.27 (t, J=5.0 Hz, 1H), 3.16-3.05 (m, 2H), 1.24 (t, J=7.2 Hz, 3H).

### Example 14

### 2-(4-Chlorophenyl)-N-propyl-benzotriazol-5-amine

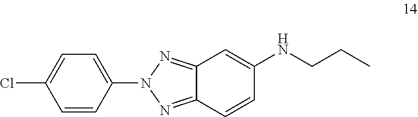 [SMILES: CCCNc1ccc2nn(-c3ccc(Cl)cc3)nc2c1]

The title compound was prepared in analogy to the procedure described for the preparation of Example 12, by using 1-bromopropane instead of iodomethane. The product was purified by preparative HPLC to afford Example 14. MS obsd. (ESI⁺) [(M+H)⁺]: 287.0. ¹H NMR (400 MHz, DMSO-d₆) δ ppm: 8.17 (d, J=8.9 Hz, 2H), 7.73-7.59 (m, 3H), 7.05 (dd, J=1.8, 9.3 Hz, 1H), 6.46 (d, J=1.2 Hz, 1H), 6.31 (br t, J=5.0 Hz, 1H), 3.09-2.97 (m, 2H), 1.64 (m, 2H), 0.98 (t, J=7.4 Hz, 3H).

### Example 15

### 2-(4-Chlorophenyl)-N-(2-methoxyethyl)benzotriazol-5-amine

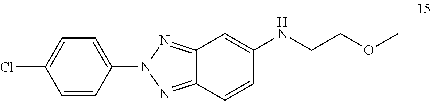 [SMILES: COCCNc1ccc2nn(-c3ccc(Cl)cc3)nc2c1]

The title compound was prepared in analogy to the procedure described for the preparation of Example 12, by using 1-bromo-2-methoxy-ethane instead of iodomethane. The product was purified by preparative HPLC to afford Example 15. MS obsd. (ESI⁺) [(M+H)⁺]: 303.1. ¹H NMR (400 MHz, DMSO-d₆) δ ppm: 8.1-8.2 (m, 2H), 7.6-7.7 (m, 3H), 7.09 (dd, 1H, J=2.0, 9.3 Hz), 6.55 (d, 1H, J=1.5 Hz), 6.34 (t, 1H, J=5.3 Hz), 3.57 (t, 2H, J=5.5 Hz), 3.31 (s, 3H), 3.2-3.3 (m, 2H).

### Example 16

### N-cyclopentyl-2-phenyl-benzotriazol-5-amine

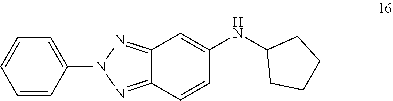 [SMILES: c1ccc(-n2nc3ccc(NC4CCCC4)cc3n2)cc1]

The title compound was prepared in analogy to the procedure described for the preparation of Example 12, by using bromocyclopentane instead of iodomethane & 2-phenylbenzotriazol-5-amine (prepared in analogy to the procedure described for the preparation of Example 1 by using aniline instead of 3-chloroaniline) instead of Example 2. The product was purified by preparative HPLC to afford Example 16. MS obsd. (ESI⁺) [(M+H)⁺]: 279.1. ¹H NMR (400 MHz, CDCl₃) δ ppm: 8.30-8.28 (m, 2H) 7.68 (d, J=8.8 Hz, 1H) 7.55-7.38 (m, 3H) 6.81-6.74 (m, 2H) 3.90-3.84 (m, 1H) 2.12-2.09 (m, 2H) 1.77-1.55 (m, 6H).

### Example 17

### N-(2-methoxyethyl)-2-(3-methoxyphenyl)benzotriazol-5-amine

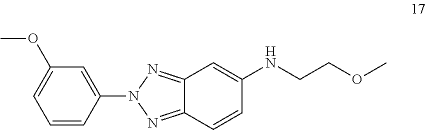

The title compound was prepared in analogy to the procedure described for the preparation of Example 12, by using 1-bromo-2-methoxy-ethane instead of iodomethane & Example 6 instead of Example 2. The product was purified by preparative HPLC to afford Example 17. MS obsd. (ESI⁺) [(M+H)⁺]: 299.1. ¹H NMR (400 MHz, DMSO-d₆) δ ppm: 7.77 (ddd, J=0.8, 1.9, 8.1 Hz, 1H), 7.74-7.65 (m, 2H), 7.50 (t, J=8.2 Hz, 1H), 7.09 (dd, J=2.1, 9.3 Hz, 1H), 7.03 (ddd, J=0.8, 2.5, 8.3 Hz, 1H), 6.57 (d, J=1.6 Hz, 1H), 6.32 (t, J=5.4 Hz, 1H), 3.88 (s, 3H), 3.58 (t, J=5.5 Hz, 2H), 3.34 (s, 3H), 3.28 (q, J=5.5 Hz, 2H).

### Example 18

### 2-(3-Chlorophenyl)-N-(2-methoxyethyl)benzotriazol-5-amine

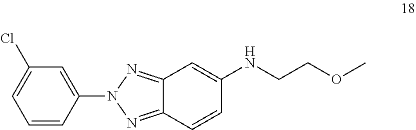 [SMILES: COCCNc1ccc2nn(-c3cccc(Cl)c3)nc2c1]

The title compound was prepared in analogy to the procedure described for the preparation of Example 12, by using 1-bromo-2-methoxy-ethane instead of iodomethane & Example 1 instead of Example 2. The product was purified by preparative HPLC to afford Example 18. MS obsd. (ESI⁺) [(M+H)⁺]: 303.1. ¹H NMR (400 MHz, DMSO-d₆) δ ppm: 8.20-8.16 (m, 1H), 8.16-8.12 (m, 1H), 7.69 (d, J=9.3 Hz, 1H), 7.66-7.59 (m, 1H), 7.52 (dd, J=1.0, 8.0 Hz, 1H), 7.11 (dd, J=2.0, 9.3 Hz, 1H), 6.54 (d, J=1.6 Hz, 1H), 6.38 (t, J=5.4 Hz, 1H), 3.57 (t, J=5.5 Hz, 2H), 3.31 (s, 3H), 3.29-3.25 (m, 2H).

### Example 19

### N-(2-methoxyethyl)-2-(4-methylsulfonylphenyl)benzotriazol-5-amine

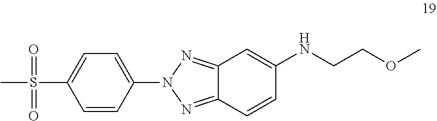

The title compound was prepared in analogy to the procedure described for the preparation of Example 12, by using 1-bromo-2-methoxy-ethane instead of iodomethane & Example 7 instead of Example 2. The product was purified by preparative HPLC to afford Example 19. MS obsd. (ESI⁺) [(M+H)⁺]: 347.0. ¹H NMR (400 MHz, DMSO-d₆) δ ppm: 8.41 (d, J=8.7 Hz, 2H), 8.13 (d, J=8.8 Hz, 2H), 7.72 (d, J=9.3 Hz, 1H), 7.14 (dd, J=1.8, 9.3 Hz, 1H), 6.55 (s, 1H), 6.46 (br t, J=5.4 Hz, 1H), 3.58 (t, J=5.5 Hz, 2H), 3.31 (br s, 3H), 3.29 (s, 2H), 2.53-2.52 (m, 3H).

### Example 20

### 2-(4-Fluorophenyl)-N-(tetrahydrofuran-3-ylmethyl)benzotriazol-5-amine

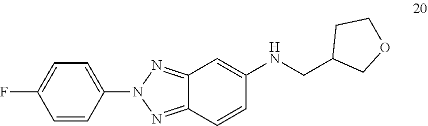 [SMILES: Fc1ccc(-n2nc3ccc(NCC4CCOC4)cc3n2)cc1]

The title compound was prepared in analogy to the procedure described for the preparation of Example 12, by using 3-(bromomethyl)tetrahydrofuran instead of iodomethane & Example 4 instead of Example 2. The product was purified by preparative HPLC to afford Example 20. MS obsd. (ESI⁺) [(M+H)⁺]: 313.1. ¹H NMR (400 MHz, MeOD-d₄) δ ppm: 8.32-8.16 (m, 2H), 7.63 (d, J=9.2 Hz, 1H), 7.31 (t, J=8.7 Hz, 2H), 7.00 (dd, J=2.0, 9.2 Hz, 1H), 6.61 (d, J=1.6 Hz, 1H), 3.99-3.88 (m, 2H), 3.80 (q, J=7.7 Hz, 1H), 3.65 (dd, J=5.6, 8.6 Hz, 1H), 3.23-3.12 (m, 2H), 2.75-2.63 (m, 1H), 2.29-2.12 (m, 1H), 1.77 (dd, J=6.0, 13.1 Hz, 1H).

### Example 21

### 2-(4-Fluorophenyl)-N-tetrahydrofuran-3-yl-benzotriazol-5-amine

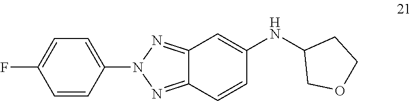 [SMILES: Fc1ccc(-n2nc3ccc(NC4CCOC4)cc3n2)cc1]

The title compound was prepared in analogy to the procedure described for the preparation of Example 12, by using 3-bromotetrahydrofuran instead of iodomethane & Example 4 instead of Example 2. The product was purified by preparative HPLC to afford Example 21. MS obsd. (ESI⁺) [(M+H)⁺]: 299.1. ¹H NMR (400 MHz, MeOD-d₄) δ ppm: 8.30-8.17 (m, 2H), 7.64 (d, J=9.3 Hz, 1H), 7.31 (t, J=8.7 Hz, 2H), 7.01 (dd, J=2.0, 9.2 Hz, 1H), 6.61 (d, J=1.8 Hz, 1H), 4.20-4.12 (m, 1H), 4.07-3.95 (m, 2H), 3.92-3.86 (m, 1H), 3.79 (dd, J=3.2, 8.9 Hz, 1H), 2.42-2.29 (m, 1H), 2.05-1.93 (m, 1H).

### Example 22

### 2-(4-Fluorophenyl)-N-(3-methoxypropyl)benzotriazol-5-amine

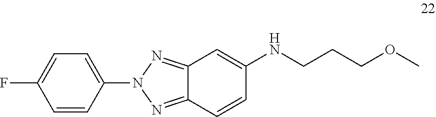 [SMILES: COCCCNc1ccc2nn(-c3ccc(F)cc3)nc2c1]

The title compound was prepared in analogy to the procedure described for the preparation of Example 12, by using 1-bromo-3-methoxy-propane instead of iodomethane & Example 4 instead of Example 2. The product was purified by preparative HPLC to afford Example 22. MS obsd. (ESI⁺) [(M+H)⁺]: 301.1. ¹H NMR (400 MHz, MeOD-d₄) δ ppm: 8.28-8.18 (m, 2H), 7.61 (d, J=9.3 Hz, 1H), 7.37-7.23 (m, 2H), 6.97 (dd, J=2.0, 9.2 Hz, 1H), 6.59 (d, J=1.7 Hz, 1H), 3.57 (t, J=6.1 Hz, 2H), 3.39 (s, 3H), 3.26 (t, J=6.8 Hz, 2H), 1.96 (t, J=6.5 Hz, 2H).

### Example 23

### 2-(4-Chlorophenyl)-N-(tetrahydrofuran-3-ylmethyl)benzotriazol-5-amine

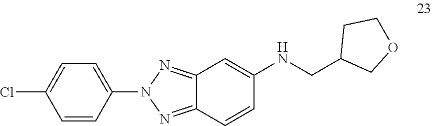 [SMILES: Clc1ccc(-n2nc3ccc(NCC4CCOC4)cc3n2)cc1]

The title compound was prepared in analogy to the procedure described for the preparation of Example 12, by using 3-(bromomethyl)tetrahydrofuran instead of iodomethane. The product was purified by preparative HPLC to afford Example 23. MS obsd. (ESI⁺) [(M+H)⁺]: 329.1. ¹H NMR (400 MHz, MeOD-d₄) δ ppm: 8.22 (d, J=9.0 Hz, 2H), 7.67-7.53 (m, 3H), 7.01 (dd, J=2.1, 9.2 Hz, 1H), 6.60 (d, J=1.7 Hz, 1H), 4.00-3.89 (m, 2H), 3.86-3.75 (m, 1H), 3.65 (dd, J=5.5, 8.6 Hz, 1H), 3.18 (dd, J=1.3, 7.4 Hz, 2H), 2.75-2.64 (m, 1H), 2.26-2.12 (m, 1H), 1.77 (dt, J=7.3, 13.1 Hz, 1H).

### Example 24

### 2-(4-Chlorophenyl)-N-(cyclobutylmethyl)benzotriazol-5-amine

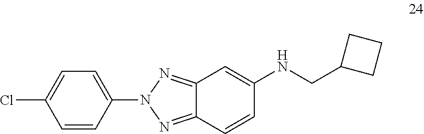 [SMILES: Clc1ccc(-n2nc3ccc(NCC4CCC4)cc3n2)cc1]

The title compound was prepared in analogy to the procedure described for the preparation of Example 12, by using bromomethylcyclobutane instead of iodomethane. The product was purified by preparative HPLC to afford Example 24. MS obsd. (ESI⁺) [(M+H)⁺]: 313.1. ¹H NMR (400 MHz, MeOD-d₄) δ ppm: 8.20-8.10 (m, 2H), 7.59 (d, J=9.3 Hz, 1H), 7.54-7.45 (m, 2H), 6.99 (d, J=9.3 Hz, 1H), 3.16 (d, J=7.2 Hz, 2H), 2.67 (td, J=7.6, 15.2 Hz, 1H), 2.17-2.08 (m, 2H), 2.01-1.75 (m, 4H).

### Example 25

### 2-(4-Chlorophenyl)-N-(cyclopropylmethyl)benzotriazol-5-amine

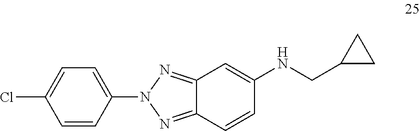 [SMILES: Clc1ccc(-n2nc3ccc(NCC4CC4)cc3n2)cc1]

The title compound was prepared in analogy to the procedure described for the preparation of Example 12, by using bromomethylcyclopropane instead of iodomethane. The product was purified by preparative HPLC to afford Example 25. MS obsd. (ESI⁺) [(M+H)⁺]: 299.1. ¹H NMR (400 MHz, MeOD-d₄) δ ppm: 8.22 (d, J=9.0 Hz, 2H), 7.64 (d, J=9.3 Hz, 1H), 7.57 (d, J=9.0 Hz, 2H), 7.10-7.01 (m, 1H), 6.63 (d, J=1.6 Hz, 1H), 3.05 (d, J=6.8 Hz, 2H), 1.24-1.13 (m, 1H), 0.68-0.56 (m, 2H), 0.38-0.24 (m, 2H).

### Example 26

### 2-(4-Chlorophenyl)-N-(cyclopentylmethyl)benzotriazol-5-amine

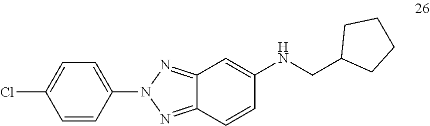 [SMILES: Clc1ccc(-n2nc3ccc(NCC4CCCC4)cc3n2)cc1]

The title compound was prepared in analogy to the procedure described for the preparation of Example 12, by using bromomethylcyclopentane instead of iodomethane. The product was purified by preparative HPLC to afford Example 26. MS obsd. (ESI⁺) [(M+H)⁺]: 327.1. ¹H NMR (400 MHz, MeOD-d₄) δ ppm: 8.21 (d, J=9.0 Hz, 2H), 7.66-7.51 (m, 3H), 7.02 (dd, J=2.1, 9.3 Hz, 1H), 6.54 (d, J=1.7 Hz, 1H), 3.08 (d, J=7.2 Hz, 2H), 2.37-2.24 (m, 1H), 1.98-1.85 (m, 2H), 1.77-1.57 (m, 4H), 1.37 (br dd, J=7.3, 12.3 Hz, 2H).

### Example 27

### 2-(4-Chlorophenyl)-N-(tetrahydrofuran-2-ylmethyl)benzotriazol-5-amine

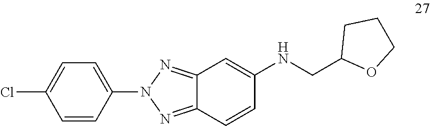 [SMILES: Clc1ccc(-n2nc3ccc(NCC4CCCO4)cc3n2)cc1]

The title compound was prepared in analogy to the procedure described for the preparation of Example 12, by using 2-(bromomethyl)tetrahydrofuran instead of iodomethane. The product was purified by preparative HPLC to afford Example 27. MS obsd. (ESI⁺) [(M+H)⁺]: 329.1. ¹H NMR (400 MHz, MeOD-d₄) δ ppm: 8.23 (dd, J=6.5, 8.9 Hz, 2H), 7.73-7.62 (m, 1H), 7.61-7.51 (m, 2H), 7.17-6.99 (m, 1H), 4.21 (dd, J=4.6, 6.9 Hz, 1H), 4.28-3.75 (m, 2H), 3.29-3.19 (m, 1H), 3.09 (d, J=6.8 Hz, 1H), 2.20-1.67 (m, 2H), 1.26-1.09 (m, 1H), 0.68-0.57 (m, 1H), 0.39-0.29 (m, 1H).

### Example 28

### 2-(4-Chlorophenyl)-N-(2,2,2-trifluoroethyl)benzotriazol-5-amine

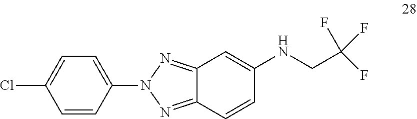 [SMILES: FC(F)(F)CNc1ccc2nn(-c3ccc(Cl)cc3)nc2c1]

The title compound was prepared in analogy to the procedure described for the preparation of Example 12, by using 2-bromo-1,1,1-trifluoro-ethane instead of iodomethane. The product was purified by preparative HPLC to afford Example 28. MS obsd. (ESI⁺) [(M+H)⁺]: 327.1. ¹H NMR (400 MHz, DMSO-d₆) δ ppm: 8.21 (d, J=9.0 Hz, 2H), 7.77 (d, J=9.2 Hz, 1H), 7.71-7.63 (m, 2H), 7.13 (dd, J=2.1, 9.2 Hz, 1H), 6.92 (s, 1H), 6.83 (t, J=6.8 Hz, 1H), 4.08 (br dd, J=6.8, 9.8 Hz, 2H).

### Example 29

### 2-(4-Chlorophenyl)-N-cyclobutyl-benzotriazol-5-amine

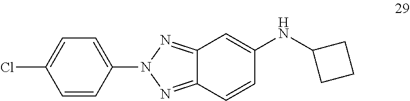 [SMILES: Clc1ccc(-n2nc3ccc(NC4CCC4)cc3n2)cc1]

The title compound was prepared in analogy to the procedure described for the preparation of Example 12, by using bromocyclobutane instead of iodomethane. The product was purified by preparative HPLC to afford Example 29. MS obsd. (ESI⁺) [(M+H)⁺]: 299.1.

### Example 30

### 2-(4-Chlorophenyl)-N-isopropyl-benzotriazol-5-amine

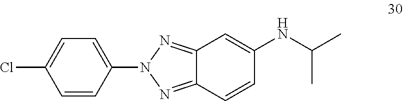 [SMILES: CC(C)Nc1ccc2nn(-c3ccc(Cl)cc3)nc2c1]

The title compound was prepared in analogy to the procedure described for the preparation of Example 12, by using 2-bromopropane instead of iodomethane. The product was purified by preparative HPLC to afford Example 30. MS obsd. (ESI⁺) [(M+H)⁺]: 287.1.

### Example 31

### 2-(4-Chlorophenyl)-N-cyclopentyl-benzotriazol-5-amine

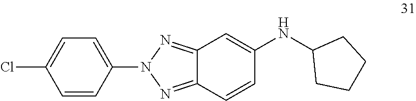 [SMILES: Clc1ccc(-n2nc3ccc(NC4CCCC4)cc3n2)cc1]

The title compound was prepared in analogy to the procedure described for the preparation of Example 12, by using bromocyclopentane instead of iodomethane. The product was purified by preparative HPLC to afford Example 31. MS obsd. (ESI⁺) [(M+H)⁺]: 313.1.

### Example 32

### 2-(4-Chlorophenyl)-N-(2,2,2-trifluoro-1-methyl-ethyl)benzotriazol-5-amine

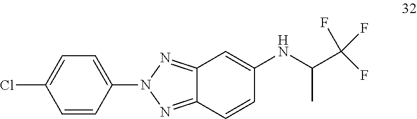 [SMILES: CC(Nc1ccc2nn(-c3ccc(Cl)cc3)nc2c1)C(F)(F)F]

The title compound was prepared in analogy to the procedure described for the preparation of Example 12, by using 2-bromo-1,1,1-trifluoro-propane instead of iodomethane. The product was purified by preparative HPLC to afford Example 32. MS obsd. (ESI⁺) [(M+H)⁺]: 341.1.

### Example 33

### 2-(4-Chlorophenyl)-N,N-dimethyl-benzotriazol-5-amine

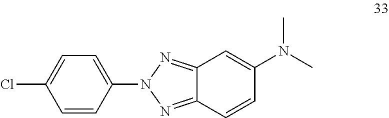 [SMILES: CN(C)c1ccc2nn(-c3ccc(Cl)cc3)nc2c1]

The title compound was prepared in analogy to the procedure described for the preparation of Example 12, by using iodomethane (4 eq) instead of iodomethane (2 eq). The product was purified by preparative HPLC to afford Example 33. MS obsd. (ESI⁺) [(M+H)⁺]: 273.1. ¹H NMR (400 MHz, DMSO-d₆) δ ppm: 8.20 (d, J=9.2 Hz, 2H), 7.81 (d, J=9.6 Hz, 1H), 7.68 (d, J=8.8 Hz, 2H), 7.34 (d, J=9.2 Hz, 1H), 6.79 (d, J=2.0 Hz, 1H), 3.03 (s, 6H).

### Example 34

### 2-(4-Chlorophenyl)-N,N-diethyl-benzotriazol-5-amine

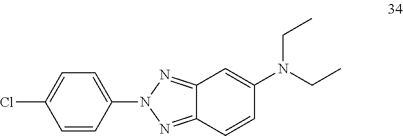 [SMILES: CCN(CC)c1ccc2nn(-c3ccc(Cl)cc3)nc2c1]

The title compound was prepared in analogy to the procedure described for the preparation of Example 12, by using bromoethane (4 eq) instead of iodomethane (2 eq). The product was purified by preparative HPLC to afford Example 34. MS obsd. (ESI⁺) [(M+H)⁺]: 301.0. ¹H NMR (400 MHz, DMSO-d₆) δ ppm: 8.31-8.17 (m, 2H), 7.81 (d, J=9.5 Hz, 1H), 7.72-7.65 (m, 2H), 7.27 (dd, J=2.3, 9.4 Hz, 1H), 6.74 (d, J=2.1 Hz, 1H), 3.46 (q, J=7.0 Hz, 4H), 1.16 (t, J=7.0 Hz, 6H).

### Example 35

### 2-(4-Chlorophenyl)-N-(2-methoxyethyl)-N-methyl-benzotriazol-5-amine

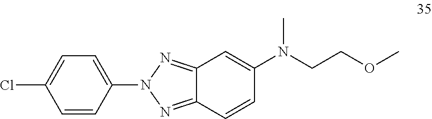 [SMILES: COCCN(C)c1ccc2nn(-c3ccc(Cl)cc3)nc2c1]

The title compound was prepared in analogy to the procedure described for the preparation of Example 12, by using 1-bromo-2-methoxy-ethane instead of iodomethane & Example 12 instead of Example 2. The product was purified by preparative HPLC to afford Example 35. MS obsd. (ESI⁺) [(M+H)⁺]: 317.0. ¹H NMR (400 MHz, DMSO-d₆) δ ppm: 8.26-8.17 (m, 2H), 7.79 (d, J=9.5 Hz, 1H), 7.66 (dd, J=1.8, 8.8 Hz, 2H), 7.34 (br d, J=9.5 Hz, 1H), 6.76 (s, 1H), 3.67-3.59 (m, 2H), 3.56-3.47 (m, 2H), 3.32 (s, 1H), 3.02 (s, 3H).

### Example 36

### 2-(4-Chlorophenyl)-N-ethyl-N-methyl-benzotriazol-5-amine

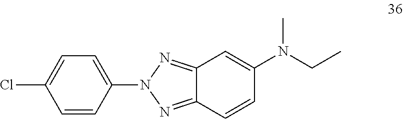 [SMILES: CCN(C)c1ccc2nn(-c3ccc(Cl)cc3)nc2c1]

The title compound was prepared in analogy to the procedure described for the preparation of Example 12, by using bromoethane instead of iodomethane & Example 12 instead of Example 2. The product was purified by preparative HPLC to afford Example 36. MS obsd. (ESI⁺) [(M+H)⁺]: 287.2. ¹H NMR (400 MHz, DMSO-d₆) δ ppm: 8.22 (d, J=8.8 Hz, 2H), 7.82 (d, J=9.5 Hz, 1H), 7.68 (d, J=8.9 Hz, 2H), 7.34 (dd, J=2.3, 9.5 Hz, 1H), 6.76 (d, J=2.0 Hz, 1H), 3.53 (q, J=7.0 Hz, 2H), 2.98 (s, 3H), 1.10 (t, J=7.0 Hz, 3H).

### Example 37

### 3-[[2-(4-Chlorophenyl)benzotriazol-5-yl]amino]-N-methyl-propanamide

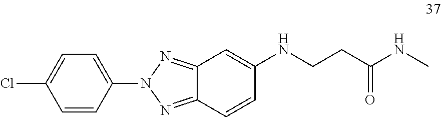 [SMILES: CNC(=O)CCNc1ccc2nn(-c3ccc(Cl)cc3)nc2c1]

The title compound was prepared according to the following scheme:

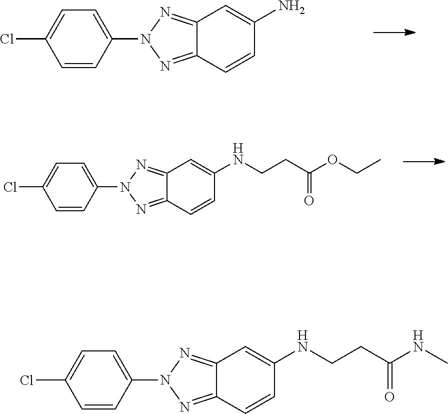 [SMILES: Nc1ccc2nn(-c3ccc(Cl)cc3)nc2c1 ; CCOC(=O)CCNc1ccc2nn(-c3ccc(Cl)cc3)nc2c1 ; CNC(=O)CCNc1ccc2nn(-c3ccc(Cl)cc3)nc2c1]

### Step 1: Preparation of ethyl 3-[[2-(4-chlorophenyl)benzotriazol-5-yl]amino]propanoate

Sodium cyanoborohydride (1071.5 mg, 17.05 mmol, 1.8 eq) was added to a suspension of 2-(4-chlorophenyl)benzotriazol-5-amine (Example 2, 2317.8 mg, 9.47 mmol, 1.0 eq), ethyl 3-oxopropanoate (1100.0 mg, 9.47 mmol, 1.0 eq) and acetic acid (1706.61 mg, 28.42 mmol, 3 eq) in methanol (80 mL). The mixture was then stirred at 20° C. for 12 h. After the reaction was completed, the solvent was removed and the residue was suspended in EA (200 mL), washed with saturated NaHCO₃ (50 mL). The separated EA layer was concentrated and purified by column chromatography eluted with (PE/EA 20/1 to 4/1) to afford ethyl 3-[[2-(4-chlorophenyl)benzotriazol-5-yl]amino]propanoate (1.12 g, 3.25 mmol, 34.2% yield) as a light yellow solid.

### Step 2: Preparation of 3-[[2-(4-chlorophenyl)benzotriazol-5-yl]amino]-N-methyl-propanamide

A mixture of ethyl 3-[[2-(4-chlorophenyl)benzotriazol-5-yl]amino]propanoate (500.0 mg, 1.45 mmol, 1 eq) in monomethylamine/ethanol (10.0 mL, 1.45 mmol, 1 eq) was stirred at 80° C. for 12 h. After the reaction completed, the resulting mixture was concentrated to give crude product, which was purified by column chromatography eluted with (DCM/MeOH, 200/1 to 50/1) to afford 3-[[2-(4-chlorophenyl)benzotriazol-5-yl]amino]-N-methyl-propanamide (250 mg, 0.76 mmol, 52.2% yield) as an off-white solid. MS obsd. (ESI⁺) [(M+H)⁺]: 330.1. ¹H NMR (400 MHz, DMSO-d₆) δ ppm: 8.27-8.14 (m, 2H), 7.87 (br d, J=4.6 Hz, 1H), 7.73-7.59 (m, 3H), 7.03 (dd, J=2.0, 9.2 Hz, 1H), 6.55 (d, J=1.6 Hz, 1H), 6.36 (t, J=5.6 Hz, 1H), 2.60 (d, J=4.6 Hz, 3H), 2.53 (d, J=1.8 Hz, 2H), 2.43 (t, J=7.0 Hz, 2H).

### Example 38

### N-[2-(4-chlorophenyl)benzotriazol-5-yl]acetamide

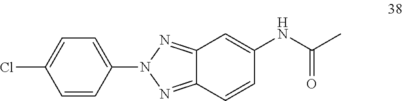 [SMILES: CC(=O)Nc1ccc2nn(-c3ccc(Cl)cc3)nc2c1]

The title compound was prepared according to the following scheme:

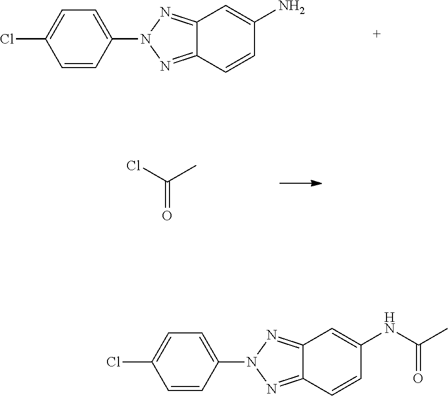

### Preparation of N-[2-(4-chlorophenyl)benzotriazol-5-yl]acetamide

The mixture of 2-(4-chlorophenyl)benzotriazol-5-amine (Example 2, 50.0 mg, 0.20 mmol, 1 eq), acetyl chloride (0.02 mL, 0.31 mmol, 1.5 eq) and triethylamine (0.06 mL, 0.41 mmol, 2 eq) in DCM (5 mL) was stirred at 20° C. for 3 h. After the start materials was consumed, the solvent was removed and the residue was purified by preparative HPLC to afford the N-[2-(4-chlorophenyl)benzotriazol-5-yl]acetamide (40 mg, 0.14 mmol, 64.1% yield). MS obsd. (ESI⁺) [(M+H)⁺]: 287.0. ¹H NMR (400 MHz, DMSO-d₆) δ ppm: 8.46 (s, 1H), 8.29 (d, J=8.8 Hz, 2H), 7.98 (d, J=9.2 Hz, 1H), 7.72 (d, J=8.8 Hz, 2H), 7.49 (d, J=9.6 Hz, 1H), 2.13 (s, 3H).

### Example 39

### N-[2-(4-chlorophenyl)benzotriazol-5-yl]cyclohexanecarboxamide

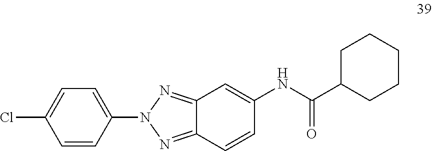 [SMILES: O=C(Nc1ccc2nn(-c3ccc(Cl)cc3)nc2c1)C1CCCCC1]

The title compound was prepared in analogy to the procedure described for the preparation of Example 38, by using cyclohexanecarbonyl chloride instead of acetyl chloride. The product was purified by preparative HPLC to afford Example 39. MS obsd. (ESI⁺) [(M+H)⁺]: 355.0. ¹H NMR (400 MHz, DMSO-d₆) δ ppm: 10.12 (s, 1H), 8.49 (d, J=0.8 Hz, 1H), 8.29 (d, J=8.8 Hz, 2H), 7.96 (d, J=9.2 Hz, 1H), 7.71 (d, J=8.8 Hz, 2H), 7.54 (d, J=9.2 Hz, 1H), 2.47-2.40 (m, 1H), 1.87-1.23 (m, 10H).

### Example 40

### N-[2-(4-fluorophenyl)benzotriazol-5-yl]tetrahydrofuran-3-carboxamide

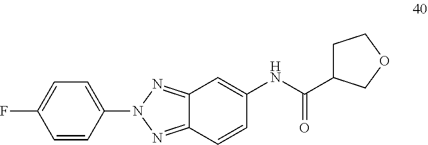 [SMILES: O=C(Nc1ccc2nn(-c3ccc(F)cc3)nc2c1)C1CCOC1]

The title compound was prepared in analogy to the procedure described for the preparation of Example 38, by using tetrahydrofuran-3-carbonyl chloride instead of acetyl chloride & Example 4 instead of Example 2. The product was purified by preparative HPLC to afford Example 40. MS obsd. (ESI⁺) [(M+H)⁺]: 327.1. ¹H NMR (400 MHz, MeOD-d₄) δ ppm: 8.40-8.30 (m, 2H), 7.89 (d, J=9.2 Hz, 1H), 7.47 (dd, J=2.0, 9.2 Hz, 1H), 7.42-7.30 (m, 2H), 4.14-4.04 (m, 1H), 4.03-3.93 (m, 2H), 3.91-3.81 (m, 1H), 3.28 (t, J=7.0 Hz, 1H), 2.34-2.22 (m, 2H).

### Example 41

### N-[2-(4-fluorophenyl)benzotriazol-5-yl]tetrahydropyran-4-carboxamide

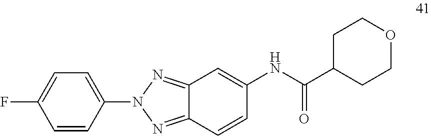 [SMILES: O=C(Nc1ccc2nn(-c3ccc(F)cc3)nc2c1)C1CCOCC1]

The title compound was prepared in analogy to the procedure described for the preparation of Example 38, by using tetrahydropyran-4-carbonyl chloride instead of acetyl chloride & Example 4 instead of Example 2. The product was purified by preparative HPLC to afford Example 41. MS obsd. (ESI⁺) [(M+H)⁺]: 341.1. ¹H NMR (400 MHz, MeOD-d₄) δ ppm: 8.48-8.32 (m, 3H), 7.89 (dd, J=0.6, 9.2 Hz, 1H), 7.48 (dd, J=1.9, 9.2 Hz, 1H), 7.42-7.30 (m, 2H), 4.05 (td, J=2.1, 9.4 Hz, 2H), 3.53 (dt, J=2.4, 11.7 Hz, 2H), 2.79-2.65 (m, 1H), 2.00-1.79 (m, 4H).

### Example 42

### N-[2-(4-fluorophenyl)benzotriazol-5-yl]tetrahydropyran-2-carboxamide

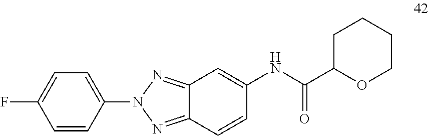 [SMILES: O=C(Nc1ccc2nn(-c3ccc(F)cc3)nc2c1)C1CCCCO1]

The title compound was prepared in analogy to the procedure described for the preparation of Example 38, by using tetrahydropyran-2-carbonyl chloride instead of acetyl chloride & Example 4 instead of Example 2. The product was purified by preparative HPLC to afford Example 42. MS obsd. (ESI⁺) [(M+H)⁺]: 341.1. ¹H NMR (400 MHz, MeOD-d₄) δ ppm: 9.57 (s, 1H), 8.49-8.32 (m, 3H), 7.90 (d, J=9.2 Hz, 1H), 7.56 (dd, J=1.9, 9.2 Hz, 1H), 7.37 (t, J=8.7 Hz, 2H), 4.19 (dd, J=3.7, 11.4 Hz, 1H), 4.01 (dd, J=2.5, 11.2 Hz, 1H), 3.64 (dt, J=2.9, 11.3 Hz, 1H), 2.20-2.07 (m, 1H), 2.04-1.93 (m, 1H), 1.77-1.51 (m, 4H).

### Example 43

### N-[2-(4-fluorophenyl)benzotriazol-5-yl]cyclopentanecarboxamide

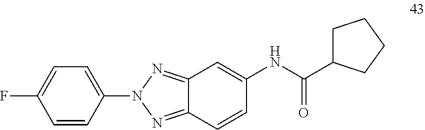 [SMILES: O=C(Nc1ccc2nn(-c3ccc(F)cc3)nc2c1)C1CCCC1]

The title compound was prepared in analogy to the procedure described for the preparation of Example 38, by using cyclopentanecarbonyl chloride instead of acetyl chloride & Example 4 instead of Example 2. The product was purified by preparative HPLC to afford Example 43. MS obsd. (ESI⁺) [(M+H)⁺]: 325.1. ¹H NMR (400 MHz, MeOD-d₄) δ ppm: 8.44 (d, J=1.2 Hz, 1H), 8.41-8.31 (m, 2H), 7.89 (d, J=9.2 Hz, 1H), 7.48 (dd, J=2.0, 9.2 Hz, 1H), 7.42-7.31 (m, 2H), 2.98-2.83 (m, 1H), 2.05-1.77 (m, 6H), 1.75-1.64 (m, 2H).

### Example 44

### 3,3,3-trifluoro-N-[2-(4-fluorophenyl)benzotriazol-5-yl]propanamide

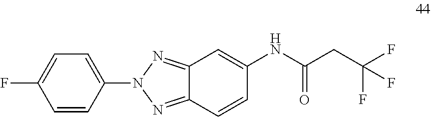 [SMILES: O=C(CC(F)(F)F)Nc1ccc2nn(-c3ccc(F)cc3)nc2c1]

The title compound was prepared in analogy to the procedure described for the preparation of Example 38, by using 3,3,3-trifluoropropanoyl chloride instead of acetyl chloride & Example 4 instead of Example 2. The product was purified by preparative HPLC to afford Example 44. MS obsd. (ESI⁺) [(M+H)⁺]: 339.1. ¹H NMR (400 MHz, MeOD-d₄) δ ppm: 8.50-8.42 (m, 1H), 8.40-8.32 (m, 2H), 7.92 (d, J=9.2 Hz, 1H), 7.45 (dd, J=1.8, 9.2 Hz, 1H), 7.40-7.30 (m, 2H), 3.44 (q, J=10.6 Hz, 2H).

### Example 45

### N-[2-(4-chlorophenyl)benzotriazol-5-yl]tetrahydropyran-4-carboxamide

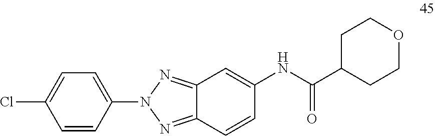 [SMILES: O=C(Nc1ccc2nn(-c3ccc(Cl)cc3)nc2c1)C1CCOCC1]

The title compound was prepared in analogy to the procedure described for the preparation of Example 38, by using tetrahydropyran-4-carbonyl chloride instead of acetyl chloride. The product was purified by preparative HPLC to afford Example 45. MS obsd. (ESI⁺) [(M+H)⁺]: 357.1. ¹H NMR (400 MHz, DMSO-d₆) δ ppm: 10.25 (s, 1H), 8.49 (d, J=1.1 Hz, 1H), 8.36-8.26 (m, 2H), 8.04-7.92 (m, 1H), 7.78-7.65 (m, 2H), 7.52 (dd, J=1.8, 9.2 Hz, 1H), 4.00-3.88 (m, 2H), 3.41 (br d, J=2.9 Hz, 1H), 2.71-2.59 (m, 1H), 2.07-1.90 (m, 1H), 1.82-1.63 (m, 4H).

### Example 46

### N-[2-(4-chlorophenyl)benzotriazol-5-yl]tetrahydropyran-2-carboxamide

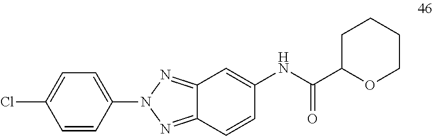 [SMILES: O=C(Nc1ccc2nn(-c3ccc(Cl)cc3)nc2c1)C1CCCCO1]

The title compound was prepared in analogy to the procedure described for the preparation of Example 38, by using tetrahydropyran-2-carbonyl chloride instead of acetyl chloride. The product was purified by preparative HPLC to afford Example 46. MS obsd. (ESI⁺) [(M+H)⁺]: 357.1. ¹H NMR (400 MHz, DMSO-d₆) δ ppm: 9.91 (s, 1H), 8.50 (d, J=1.1 Hz, 1H), 8.36-8.27 (m, 2H), 7.97 (d, J=9.2 Hz, 1H), 7.81-7.66 (m, 3H), 5.32 (t, J=4.7 Hz, 1H), 4.12-3.96 (m, 2H), 3.61-3.49 (m, 1H), 2.09-1.92 (m, 3H), 1.56 (br d, J=4.2 Hz, 3H).

### Example 47

### N-[2-(4-chlorophenyl)benzotriazol-5-yl]-1,4-dioxane-2-carboxamide

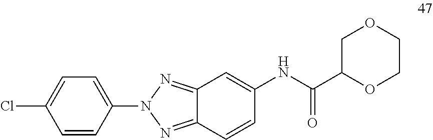 [SMILES: O=C(Nc1ccc2nn(-c3ccc(Cl)cc3)nc2c1)C1COCCO1]

The title compound was prepared in analogy to the procedure described for the preparation of Example 38, by using 1,4-dioxane-2-carbonyl chloride instead of acetyl chloride. The product was purified by preparative HPLC to afford Example 47. MS obsd. (ESI⁺) [(M+H)⁺]: 359.1. ¹H NMR (400 MHz, DMSO-d₆) δ ppm: 10.13 (s, 1H), 8.48 (d, J=1.0 Hz, 1H), 8.35-8.29 (m, 2H), 7.99 (d, J=9.3 Hz, 1H), 7.79-7.67 (m, 3H), 4.31 (dd, J=3.1, 9.2 Hz, 1H), 4.04-3.89 (m, 2H), 3.82-3.69 (m, 2H), 3.66-3.54 (m, 2H).

### Example 48

### N-[2-(4-chlorophenyl)benzotriazol-5-yl]tetrahydrofuran-3-carboxamide

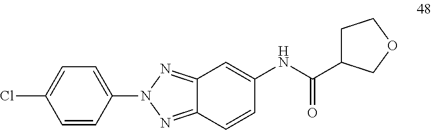 [SMILES: O=C(Nc1ccc2nn(-c3ccc(Cl)cc3)nc2c1)C1CCOC1]

The title compound was prepared in analogy to the procedure described for the preparation of Example 38, by using tetrahydrofuran-3-carbonyl chloride instead of acetyl chloride. The product was purified by preparative HPLC to afford Example 48. MS obsd. (ESI⁺) [(M+H)⁺]: 343.1. ¹H NMR (400 MHz, DMSO-d₆) δ ppm: 10.38 (s, 1H), 8.48 (d, J=1.2 Hz, 1H), 8.36-8.26 (m, 2H), 7.99 (dd, J=0.7, 9.2 Hz, 1H), 7.78-7.67 (m, 2H), 7.52 (dd, J=1.9, 9.2 Hz, 1H), 3.97 (t, J=8.3 Hz, 1H), 3.84-3.66 (m, 3H), 3.26-3.14 (m, 1H), 2.13 (q, J=7.1 Hz, 2H).

### Example 49

### N-[2-(4-chlorophenyl)benzotriazol-5-yl]cyclopentanecarboxamide

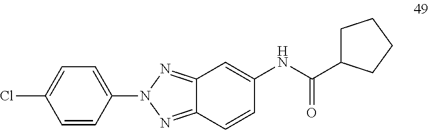 [SMILES: O=C(Nc1ccc2nn(-c3ccc(Cl)cc3)nc2c1)C1CCCC1]

The title compound was prepared in analogy to the procedure described for the preparation of Example 38, by using cyclopentanecarbonyl chloride instead of acetyl chloride. The product was purified by preparative HPLC to afford Example 49. MS obsd. (ESI⁺) [(M+H)⁺]: 341.1. ¹H NMR (400 MHz, DMSO-d₆) δ ppm: 10.21 (s, 1H), 8.49 (d, J=1.1 Hz, 1H), 8.26-8.32 (m, 2H), 7.97 (d, J=9.2 Hz, 1H), 7.68-7.73 (m, 2H), 7.53 (dd, J=9.3, 1.8 Hz, 1H), 2.85 (quin, J=7.9 Hz, 1H), 1.84-1.94 (m, 2H), 1.54-1.81 (m, 6H).

### Example 50

### N-[2-(4-chlorophenyl)benzotriazol-5-yl]tetrahydrofuran-2-carboxamide

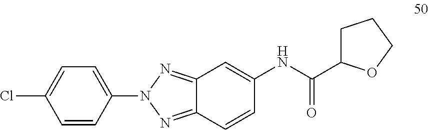 [SMILES: O=C(Nc1ccc2nn(-c3ccc(Cl)cc3)nc2c1)C1CCCO1]

The title compound was prepared in analogy to the procedure described for the preparation of Example 38, by using tetrahydrofuran-2-carbonyl chloride instead of acetyl chloride. The product was purified by preparative HPLC to afford Example 50. MS obsd. (ESI⁺) [(M+H)⁺]: 343.1. ¹H NMR (400 MHz, DMSO-d₆) δ ppm: 10.03 (s, 1H), 8.50 (d, J=1.2 Hz, 1H), 8.30 (d, J=8.1 Hz, 2H), 7.98 (d, J=9.2 Hz, 1H), 7.69-7.75 (m, 3H), 4.47 (dd, J=8.2, 5.6 Hz, 1H), 3.99-4.05 (m, 1H), 3.83-3.91 (m, 1H), 2.18-2.28 (m, 1H), 1.85-2.09 (m, 3H).

### Example 51

### N-[2-(4-chlorophenyl)benzotriazol-5-yl]-3,3,3-trifluoro-propanamide

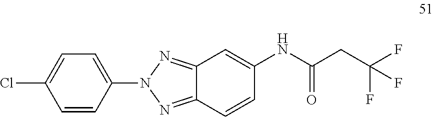 [SMILES: O=C(CC(F)(F)F)Nc1ccc2nn(-c3ccc(Cl)cc3)nc2c1]

The title compound was prepared in analogy to the procedure described for the preparation of Example 38, by using 3,3,3-trifluoropropanoyl chloride instead of acetyl chloride. The product was purified by preparative HPLC to afford Example 51. MS obsd. (ESI⁺) [(M+H)⁺]: 355.0. ¹H NMR (400 MHz, DMSO-d₆) δ ppm: 10.66 (s, 1H), 8.45 (d, J=1.2 Hz, 1H), 8.36-8.26 (m, 2H), 8.04 (dd, J=0.6, 9.2 Hz, 1H), 7.79-7.69 (m, 2H), 7.48 (dd, J=1.9, 9.2 Hz, 1H), 3.61 (q, J=11.1 Hz, 2H).

### Example 52

### N-[2-(4-chlorophenyl)benzotriazol-5-yl]-4,4,4-trifluoro-butanamide

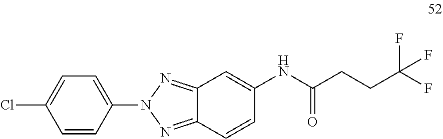 [SMILES: O=C(CCC(F)(F)F)Nc1ccc2nn(-c3ccc(Cl)cc3)nc2c1]

The title compound was prepared in analogy to the procedure described for the preparation of Example 38, by using 4,4,4-trifluorobutanoyl chloride instead of acetyl chloride. The product was purified by preparative HPLC to afford Example 52. MS obsd. (ESI⁺) [(M+H)⁺]: 369.0. ¹H NMR (400 MHz, DMSO-d₆) δ ppm: 10.44 (s, 1H), 8.47 (d, J=1.1 Hz, 1H), 8.36-8.24 (m, 2H), 8.06-7.95 (m, 1H), 7.77-7.67 (m, 2H), 7.49 (dd, J=1.8, 9.3 Hz, 1H), 2.78-2.60 (m, 4H).

### Example 53

### [2-(4-Chlorophenyl)benzotriazol-5-yl]thiourea

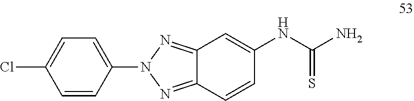 [SMILES: NC(=S)Nc1ccc2nn(-c3ccc(Cl)cc3)nc2c1]

The title compound was prepared according to the following scheme:

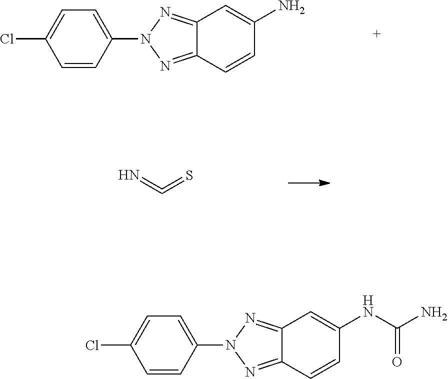

### Preparation of [2-(4-chlorophenyl)benzotriazol-5-yl]thiourea

To a solution of 2-(4-chlorophenyl)benzotriazol-5-amine (Example 2, 80.0 mg, 0.33 mmol, 1 eq) in THF (1 mL) was added isothiocyanic acid (20 mg, 0.33 mmol, 1 eq). The mixture was then stirred at 25° C. for 16 h. After the reaction completed, the solvent was removed and the residue was purified by preparative HPLC to afford Example 53. MS obsd. (ESI⁺) [(M+H)⁺]: 304.0. ¹H NMR (400 MHz, DMSO-d₆) δ ppm: 10.02 (s, 1H), 8.33-8.26 (m, 3H), 7.97 (d, J=9.6 Hz, 1H), 7.72 (d, J=9.2 Hz, 2H), 7.48 (d, J=9.2 Hz, 1H).

### Example 54

### 1-[2-(4-chlorophenyl)benzotriazol-5-yl]-3-methyl-thiourea

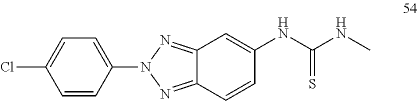 [SMILES: CNC(=S)Nc1ccc2nn(-c3ccc(Cl)cc3)nc2c1]

The title compound was prepared in analogy to the procedure described for the preparation of Example 53, by using methylimino(thioxo)methane instead of isothiocyanic acid. The product was purified by preparative HPLC to afford Example 54. MS obsd. (ESI⁺) [(M+H)⁺]: 318.0. ¹H NMR (400 MHz, DMSO-d₆) δ ppm: 9.91 (s, 1H), 8.30 (d, J=8.8 Hz, 2H), 8.18 (s, 1H), 7.96-7.95 (m, 2H), 7.72 (d, J=9.2 Hz, 2H), 7.48 (s, 1H), 2.96 (d, J=3.6 Hz, 3H).

### Example 55

### N-[[2-(4-chlorophenyl)benzotriazol-5-yl]carbamothioyl]acetamide

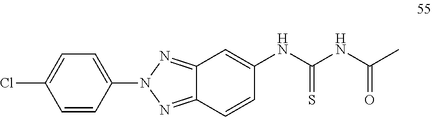 [SMILES: CC(=O)NC(=S)Nc1ccc2nn(-c3ccc(Cl)cc3)nc2c1]

The title compound was prepared in analogy to the procedure described for the preparation of Example 53, by using acetyl isothiocyanate instead of isothiocyanic acid. The product was purified by preparative HPLC to afford Example 55. MS obsd. (ESI⁺) [(M+H)⁺]: 346.0. ¹H NMR (400 MHz, DMSO-d₆) δ ppm: 12.69 (s, 1H), 11.63 (s, 1H), 8.55 (s, 1H), 8.32 (d, J=9.2 Hz, 2H), 8.03 (d, J=9.2 Hz, 1H), 7.73 (d, J=9.2 Hz, 2H), 7.56 (d, J=6.8 Hz, 1H), 2.20 (s, 3H).

### Example 56

### N-[[2-(4-chlorophenyl)benzotriazol-5-yl]carbamothioyl]cyclopentanecarboxamide

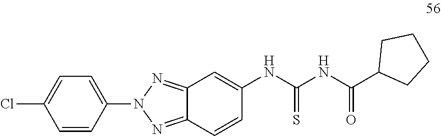 [SMILES: O=C(NC(=S)Nc1ccc2nn(-c3ccc(Cl)cc3)nc2c1)C1CCCC1]

The title compound was prepared in analogy to the procedure described for the preparation of Example 53, by using cyclopentanecarbonyl isothiocyanate instead of isothiocyanic acid. The product was purified by preparative HPLC to afford Example 56. MS obsd. (ESI⁺) [(M+H)⁺]: 400.1. ¹H NMR (400 MHz, DMSO-d₆) δ ppm: 12.74 (s, 1H), 11.59 (s, 1H), 8.56 (s, 1H), 8.32 (d, J=9.2 Hz, 2H), 8.03 (d, J=9.2 Hz, 1H), 7.73 (d, J=9.2 Hz, 2H), 7.58 (d, J=6.8 Hz, 1H), 3.07-3.01 (m, 1H), 1.91-1.57 (m, 8H).

### Example 57

### N-[[2-(4-chlorophenyl)benzotriazol-5-yl]carbamothioyl]-2-methyl-propanamide

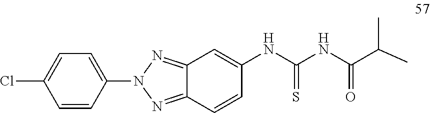 [SMILES: CC(C)C(=O)NC(=S)Nc1ccc2nn(-c3ccc(Cl)cc3)nc2c1]

The title compound was prepared in analogy to the procedure described for the preparation of Example 53, by using 2-methylpropanoyl isothiocyanate instead of isothiocyanic acid. The product was purified by preparative HPLC to afford Example 57. MS obsd. (ESI⁺) [(M+H)⁺]: 374.0. ¹H NMR (400 MHz, DMSO-d₆) δ ppm: 12.73 (s, 1H), 11.60 (s, 1H), 8.56 (s, 1H), 8.32 (d, J=8.8 Hz, 2H), 8.03 (d, J=9.2 Hz, 1H), 7.73 (d, J=9.2 Hz, 2H), 7.58 (d, J=9.2 Hz, 1H), 2.90-2.80 (m, 1H), 1.13 (d, J=6.8 Hz, 6H).

### Example 58

### N-[[2-(4-chlorophenyl)benzotriazol-5-yl]carbamothioyl]benzamide

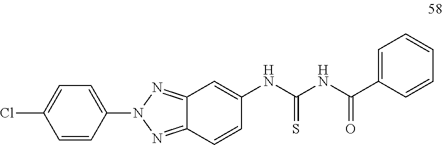 [SMILES: O=C(NC(=S)Nc1ccc2nn(-c3ccc(Cl)cc3)nc2c1)c1ccccc1]

The title compound was prepared in analogy to the procedure described for the preparation of Example 53, by using benzoyl isothiocyanate instead of isothiocyanic acid. The product was purified by preparative HPLC to afford Example 58. MS obsd. (ESI⁺) [(M+H)⁺]: 408.1. ¹H NMR (400 MHz, DMSO-d₆) δ ppm: 12.78 (s, 1H), 11.73 (s, 1H), 8.59 (s, 1H), 8.34 (d, J=12.0 Hz, 2H), 8.05-8.00 (m, 3H), 7.76-7.56 (m, 6H).

### Example 59

### N-[[2-(4-chlorophenyl)-6-methyl-benzotriazol-5-yl]carbamothioyl]benzamide

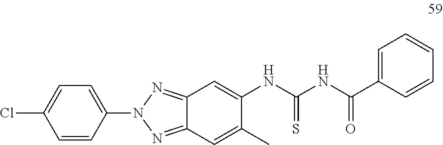 [SMILES: Cc1cc2nn(-c3ccc(Cl)cc3)nc2cc1NC(=S)NC(=O)c1ccccc1]

The title compound was prepared in analogy to the procedure described for the preparation of Example 53, by using benzoyl isothiocyanate instead of isothiocyanic acid & 2-(4-chlorophenyl)-6-methyl-benzotriazol-5-amine (Prepared in in analogy to the procedure described for the preparation of Example 1, by using 4-chloroaniline instead of 3-chloroaniline & 4-methylbenzene-1,3-diamine instead of benzene-1,3-diamine) instead of Example 2. The product was purified by preparative HPLC to afford Example 59. MS obsd. (ESI⁺) [(M+H)⁺]: 443.9. ¹H NMR (400 MHz, DMSO-d₆) δ ppm: 8.44 (s, 1H), 8.32 (d, J=8.8 Hz, 2H), 8.04-7.97 (m, 3H), 7.74-7.56 (m, 5H), 2.47 (s, 3H).

### Example 60

### [2-(4-Chlorophenyl)-6-methyl-benzotriazol-5-yl]thiourea

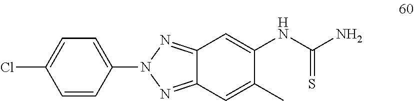 [SMILES: Cc1cc2nn(-c3ccc(Cl)cc3)nc2cc1NC(N)=S]

The title compound was prepared in analogy to the procedure described for the preparation of Example 53, by using 2-(4-chlorophenyl)-6-methyl-benzotriazol-5-amine (Prepared in in analogy to the procedure described for the preparation of Example 1, by using 4-chloroaniline instead of 3-chloroaniline & 4-methylbenzene-1,3-diamine instead of benzene-1,3-diamine) instead of Example 2. The product was purified by preparative HPLC to afford Example 60. MS obsd. (ESI⁺) [(M+H)⁺]: 318.0. ¹H NMR (400 MHz, DMSO-d₆) δ ppm: 9.46 (br, s, 1H), 8.32 (d, J=8.8 Hz, 2H), 7.89 (d, J=8.4 Hz, 2H), 7.75-7.72 (m, 2H), 2.23 (s, 3H).

### Example 61

### 3-[2-(4-Chlorophenyl)benzotriazol-5-yl]-1,1-dimethyl-thiourea

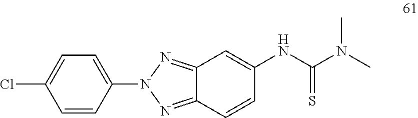 [SMILES: CN(C)C(=S)Nc1ccc2nn(-c3ccc(Cl)cc3)nc2c1]

The title compound was prepared according to the following scheme:

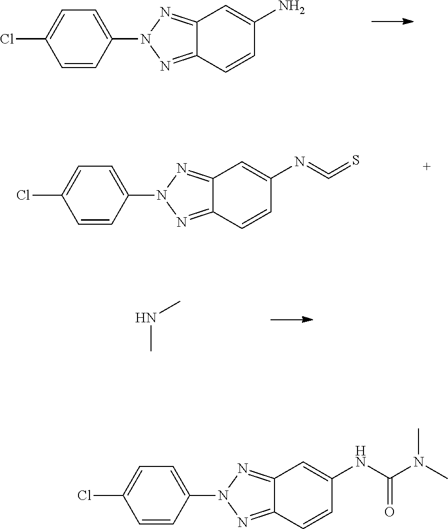

### Step 1: Preparation of 2-(4-chlorophenyl)-5-isothiocyanato-benzotriazole

A mixture of 2-(4-chlorophenyl)benzotriazol-5-amine (Example 2, 300.0 mg, 1.23 mmol, 1 eq) and 1,1′-thiocarbonyldiimidazole (262.2 mg, 1.47 mmol, 1.2 eq) in THF (1 mL) was stirred at 25° C. for 3 h. After the reaction completed, the resulting mixture was poured into H₂O (5 mL) and extracted with EA (5 mL*3). The combined organic layer was washed with brine (5 mL), dried over Na₂SO₄, filtered and concentrated to give crude product 2-(4-chlorophenyl)-5-isothiocyanato-benzotriazole (120 mg, 0.420 mmol, 34.13% yield) as a brown solid which used in the next step without further purification.

### Step 2: Preparation of 3-[2-(4-chlorophenyl)benzotriazol-5-yl]-1,1-dimethyl-thiourea

The mixture of 2-(4-chlorophenyl)-5-isothiocyanato-benzotriazole (120.0 mg, 0.42 mmol, 1 eq) and dimethylamine (2.0 mL, 21.67 mmol, 51.78 eq) in THF (2 mL) was stirred at 20° C. for 12 h. After the reaction completed, the resulting mixture was concentrated and the residue was purified by preparative HPLC (FA) to afford Example 61 (20.7 mg, 0.060 mmol, 7.24% yield) as a brown solid. MS obsd. (ESI⁺) [(M+H)⁺]: 332.0. ¹H NMR (400 MHz, DMSO-d₆) δ ppm: 9.36 (s, 1H), 8.30 (d, J=8.8 Hz, 2H), 7.90 (d, J=9.2 Hz, 1H), 7.79 (s, 1H), 7.74-7.72 (m, 2H), 7.59 (d, J=1.6 Hz, 1H), 3.35 (s, 6H).

### Example 62

### N-[2-(4-chlorophenyl)benzotriazol-5-yl]-4-isopropyl-thiazol-2-amine

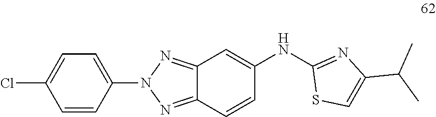 [SMILES: CC(C)c1csc(Nc2ccc3nn(-c4ccc(Cl)cc4)nc3c2)n1]

The title compound was prepared according to the following scheme:

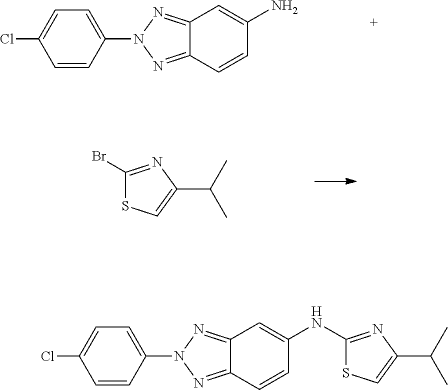

### Preparation of N-[2-(4-chlorophenyl)benzotriazol-5-yl]-4-isopropyl-thiazol-2-amine

2-(4-chlorophenyl)benzotriazol-5-amine (Example 2, 100.0 mg, 0.41 mmol, 1 eq) and 2-bromo-4-isopropylthiazole (0.07 mL, 0.61 mmol, 1.5 eq) was suspended in 1-butanol (3 mL), p-toluenesulfonic acid (35.19 mg, 0.20 mmol, 0.50 eq) was added and the mixture was stirred at 120° C. for 12 h. After the reaction completed, the resulting mixture was concentrated and the residue was purified by prep-HPLC (FA) to afford Example 62 (7.8 mg, 0.02 mmol, 4.8% yield) as a yellow solid. MS obsd. (ESI⁺) [(M+H)⁺]: 370.0. ¹H NMR (400 MHz, DMSO-d₆) δ ppm: 10.58 (s, 1H), 8.64 (d, J=1.4 Hz, 1H), 8.34-8.24 (m, 2H), 7.95 (d, J=9.5 Hz, 1H), 7.74-7.64 (m, 2H), 7.39 (dd, J=1.9, 9.3 Hz, 1H), 6.57 (d, J=0.9 Hz, 1H), 2.95 (td, J=6.7, 13.5 Hz, 1H), 1.29 (d, J=6.8 Hz, 6H).

### Example 63

### [2-(4-Chlorophenyl)-6-methyl-benzotriazol-5-yl]thiourea

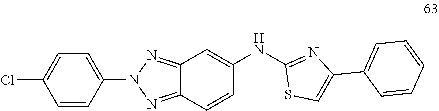 [SMILES: Clc1ccc(-n2nc3ccc(Nc4nc(-c5ccccc5)cs4)cc3n2)cc1]

The title compound was prepared in analogy to the procedure described for the preparation of Example 62, by using 2-bromo-4-phenyl-thiazole instead of 2-bromo-4-isopropylthiazole. The product was purified by preparative HPLC to afford Example 63. MS obsd. (ESI⁺) [(M+H)⁺]: 404.0. ¹H NMR (400 MHz, DMSO-d₆) δ ppm: 10.75 (s, 1H), 8.78 (d, J=1.3 Hz, 1H), 8.31 (d, J=8.9 Hz, 2H), 8.03-7.96 (m, 3H), 7.71 (d, J=8.9 Hz, 2H), 7.58-7.43 (m, 4H), 7.41-7.30 (m, 1H).

### Example 64

### N-[2-(4-chlorophenyl)benzotriazol-5-yl]-4-methyl-thiazol-2-amine

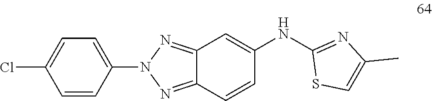 [SMILES: Cc1csc(Nc2ccc3nn(-c4ccc(Cl)cc4)nc3c2)n1]

The title compound was prepared in analogy to the procedure described for the preparation of Example 62, by using 2-bromo-4-methyl-thiazole instead of 2-bromo-4-isopropylthiazole. The product was purified by preparative HPLC to afford Example 64. MS obsd. (ESI⁺) [(M+H)⁺]: 342.1. ¹H NMR (400 MHz, DMSO-d₆) δ ppm: 10.57 (s, 1H), 8.66 (s, 1H), 8.29 (d, J=9.0 Hz, 2H), 7.95 (d, J=9.3 Hz, 1H), 7.71 (d, J=8.9 Hz, 2H), 7.39 (dd, J=1.9, 9.3 Hz, 1H), 6.59 (s, 1H), 2.32 (s, 3H).

### Example 65

### N-[2-(4-chlorophenyl)benzotriazol-5-yl]-4,5-dimethyl-thiazol-2-amine

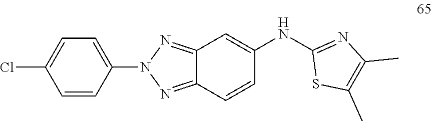 [SMILES: Cc1nc(Nc2ccc3nn(-c4ccc(Cl)cc4)nc3c2)sc1C]

The title compound was prepared in analogy to the procedure described for the preparation of Example 62, by using 2-bromo-4,5-dimethyl-thiazole instead of 2-bromo-4-isopropylthiazole. The product was purified by preparative HPLC to afford Example 65. MS obsd. (ESI⁺) [(M+H)⁺]: 356.0. ¹H NMR (400 MHz, DMSO-d₆) δ ppm: 10.37 (br s, 1H), 8.60 (d, J=1.4 Hz, 1H), 8.28 (d, J=8.9 Hz, 2H), 7.92 (d, J=9.3 Hz, 1H), 7.70 (d, J=8.9 Hz, 2H), 7.35 (dd, J=1.8, 9.2 Hz, 1H), 2.25 (s, 3H), 2.22 (s, 3H).

### Biological Examples

### Example 66

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 μl of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat. 10099141), 5 ng/mL human epidermal growth factor (Gibco, Cat.PHG031IL), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 1.

TABLE 1
Activity data of compounds of this invention

| Example No. | HBsAg IC₅₀ (μM) | HBeAg IC₅₀ (μM) | CC₅₀ (μM) |
|---|---|---|---|
| Example 1 | 1.20 | 1.80 | >100 |
| Example 2 | 7.97 | 5.29 | >100 |
| Example 3 | 1.50 | 1.70 | >100 |
| Example 4 | 5.04 | 5.29 | >100 |
| Example 5 | 3.57 | 6.72 | >100 |
| Example 6 | 2.44 | 4.10 | >100 |
| Example 7 | 5.67 | 3.21 | >100 |
| Example 8 | 6.32 | 6.26 | >100 |
| Example 9 | 4.07 | 7.93 | >100 |
| Example 10 | 9.57 | 5.56 | >100 |
| Example 11 | 2.66 | 3.89 | >100 |
| Example 12 | 1.83 | 2.44 | >100 |
| Example 13 | 1.30 | 1.60 | >100 |
| Example 14 | 2.92 | 3.97 | >100 |
| Example 15 | 3.71 | 2.36 | >100 |
| Example 16 | 9.25 | 15.76 | >100 |
| Example 17 | 8.06 | 15.44 | >100 |
| Example 18 | 4.50 | 6.83 | >100 |
| Example 19 | 8.94 | 6.97 | >100 |
| Example 20 | 5.66 | 7.30 | >100 |
| Example 21 | 9.74 | 10.55 | >100 |
| Example 22 | 3.27 | 6.40 | >100 |
| Example 23 | 4.92 | 7.67 | >100 |
| Example 24 | 7.72 | 8.90 | >100 |
| Example 25 | 5.95 | 7.52 | >100 |
| Example 26 | 8.79 | 3.08 | >100 |
| Example 27 | 6.07 | 11.69 | >100 |
| Example 28 | 2.14 | 2.46 | >100 |
| Example 29 | 5.55 | 9.23 | >100 |
| Example 30 | 3.75 | 6.61 | >100 |
| Example 31 | 8.86 | 11.86 | >100 |
| Example 32 | 9.14 | 10.73 | >100 |
| Example 33 | 4.00 | 3.71 | >100 |
| Example 34 | 1.80 | 2.50 | >100 |
| Example 35 | 2.00 | 3.50 | >100 |
| Example 36 | 4.03 | 4.45 | >100 |
| Example 37 | 4.64 | 8.68 | >100 |
| Example 38 | 3.65 | 5.54 | >100 |
| Example 39 | 3.91 | 2.89 | >100 |
| Example 40 | 6.66 | 6.76 | >100 |
| Example 41 | 2.19 | 2.31 | >100 |
| Example 42 | 6.38 | 6.12 | >100 |
| Example 43 | 8.20 | 12.79 | >100 |
| Example 44 | 6.87 | 11.68 | >100 |
| Example 45 | 3.39 | 1.98 | >100 |
| Example 46 | 2.68 | 1.14 | >100 |
| Example 47 | 3.73 | 1.84 | >100 |
| Example 48 | 4.81 | 3.12 | >100 |
| Example 49 | 2.84 | 1.29 | >100 |
| Example 50 | 1.26 | 1.37 | >100 |
| Example 51 | 4.09 | 1.66 | >100 |
| Example 52 | 2.25 | 1.89 | >100 |
| Example 53 | 3.37 | 2.87 | >100 |
| Example 54 | 2.68 | 4.59 | >100 |
| Example 55 | 2.97 | 5.10 | >100 |
| Example 56 | 3.45 | 7.50 | >100 |
| Example 57 | 2.50 | 3.72 | >100 |
| Example 58 | 1.17 | 1.27 | >100 |
| Example 59 | 1.11 | 3.04 | >100 |
| Example 60 | 2.28 | 2.31 | >100 |
| Example 61 | 0.79 | 1.22 | >100 |
| Example 62 | 3.80 | 5.80 | >100 |
| Example 63 | 1.52 | 1.34 | >100 |
| Example 64 | 1.42 | 3.04 | >100 |
| Example 65 | 2.67 | 2.91 | >100 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

We claim:

1. A compound of the formula (I), 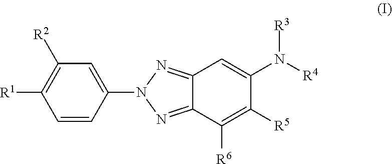
wherein
R¹ is H, halogen, C₁₋₆alkyl, C₁₋₆alkoxy, C₁₋₆alkylsulfonyl or C₁₋₆alkoxyC₁₋₆alkylsulfonyl;
R² is H, halogen, C₁₋₆alkyl, C₁₋₆alkoxy, C₁₋₆alkylsulfonyl or C₁₋₆alkoxyC₁₋₆alkylsulfonyl;
R³ is H, C₁₋₆alkyl, haloC₁₋₆alkyl, C₁₋₆alkoxyC₁₋₆alkyl, C₃₋₇cycloalkyl, C₃₋₇cycloalkylC₁₋₆alkyl, C₁₋₆alkylaminocarbonylC₁₋₆alkyl, heterocyclyl, heterocyclylC₁₋₆alkyl, C₁₋₆alkylheterocyclyl, (C₁₋₆alkyl)₂heterocyclyl, phenylheterocyclyl or —C(X)—R⁷; wherein
X is O or S;
R⁷ is amino, C₁₋₆alkyl, haloC₁₋₆alkyl, C₁₋₆alkylamino, (C₁₋₆alkyl)₂amino, C₃₋₇cycloalkyl, C₁₋₆alkylcarbonylamino, C₃₋₇cycloalkylcarbonylamino, phenylcarbonylamino or heterocyclyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

2. A compound according to claim 1, wherein
R¹ is H, halogen, C₁₋₆alkoxy, C₁₋₆alkylsulfonyl or C₁₋₆alkoxyC₁₋₆alkylsulfonyl;
R² is H, halogen or C₁₋₆alkoxy;
R³ is H, C₁₋₆alkyl, haloC₁₋₆alkyl, C₁₋₆alkoxyC₁₋₆alkyl, C₃₋₇cycloalkyl, C₃₋₇cycloalkylC₁₋₆alkyl, C₁₋₆alkylaminocarbonylC₁₋₆alkyl, tetrahydrofuranyl, tetrahydrofuranylC₁₋₆alkyl, C₁₋₆ alkylthiazolyl, (C₁₋₆alkyl)₂thiazolyl, phenylthiazolyl or —C(X)—R⁷; wherein
X is O or S;
R⁷ is amino, C₁₋₆alkyl, haloC₁₋₆alkyl, C₁₋₆alkylamino, (C₁₋₆alkyl)₂amino, C₃₋₇cycloalkyl, C₁₋₆alkylcarbonylamino, C₃₋₇cycloalkylcarbonylamino, phenylcarbonylamino, tetrahydrofuranyl, tetrahydropyranyl or 1,4-dioxanyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

3. A compound according to claim 1, wherein
R¹ is H, F, Cl, methoxy, methylsulfonyl, ethylsulfonyl, isobutylsulfonyl or methoxyethylsulfonyl;
R² is H, F, Cl or methoxy;
R³ is H, methyl, ethyl, propyl, isopropyl, trifluoroethyl, trifluoromethyletyl, methoxyethyl, methoxypropyl, cyclobutyl, cyclopentyl, cyclopropylmethyl, cyclobutylmethyl, cyclopentylmethyl, methylaminocarbonylethyl, tetrahydrofuranyl, tetrahydrofuranylmethyl, methylthiazolyl, isopropylthiazolyl, (methyl)₂thiazolyl, phenylthiazolyl or —C(X)—R⁷;
wherein
X is O or S;
R⁷ is amino, methyl, trifluoroethyl, trifluoropropyl, methylamino, dimethylamino, cyclopentyl, cyclohexyl, methylcarbonylamino, isopropylcarbonylamino, cyclopentylcarbonylamino, phenylcarbonylamino, tetrahydrofuranyl, tetrahydropyranyl or 1,4-dioxanyl;
R⁴ is H, methyl or ethyl;
R⁵ is H or methyl;
R⁶ is H or methyl;
wherein with the proviso that R¹, R² and R³ are not H simultaneously;
or a pharmaceutically acceptable salt thereof.

4. A compound according to claim 1, or a pharmaceutically acceptable salt thereof, wherein R¹ is H, halogen or C₁₋₆alkoxy.

5. A compound according to claim 4, or a pharmaceutically acceptable salt thereof, wherein
R¹ is H, F, Cl or methoxy.

6. A compound according to claim 1, or a pharmaceutically acceptable salt thereof, wherein
R³ is H, C₁₋₆alkyl, haloC₁₋₆alkyl, C₁₋₆alkoxyC₁₋₆alkyl, C₁₋₆alkylthiazolyl, (C₁₋₆alkyl)₂thiazolyl, phenylthiazolyl or —C(X)—R⁷; wherein
X is O or S;
R⁷ is amino, haloC₁₋₆alkyl, C₁₋₆alkylamino, (C₁₋₆alkyl)₂amino, C₁₋₆alkylcarbonylamino, phenylcarbonylamino or tetrahydropyranyl.

7. A compound according to claim 6, or a pharmaceutically acceptable salt thereof, wherein
R³ is H, ethyl, propyl, trifluoroethyl, methoxyethyl, methylthiazolyl, (methyl)₂thiazolyl, phenylthiazolyl or —C(X)—R⁷; wherein
X is O or S;
R⁷ is amino, trifluoropropyl, methylamino, dimethylamino, methylcarbonylamino, isopropylcarbonylamino, phenylcarbonylamino or tetrahydropyranyl.

8. A compound according to claim 1, wherein
R¹ is H, halogen or C₁₋₆alkoxy;
R² is H, halogen or C₁₋₆alkoxy;
R³ is H, C₁₋₆alkyl, haloC₁₋₆alkyl, C₁₋₆alkoxyC₁₋₆alkyl, C₁₋₆alkylthiazolyl, (C₁₋₆alkyl)₂thiazolyl, phenylthiazolyl or —C(X)—R⁷; wherein
X is O or S;
R⁷ is amino, haloC₁₋₆alkyl, C₁₋₆alkylamino, (C₁₋₆alkyl)₂amino, C₁₋₆alkylcarbonylamino, phenylcarbonylamino or tetrahydropyranyl;
R⁴ is H or C₁₋₆alkyl;
R⁵ is H or C₁₋₆alkyl;
R⁶ is H or C₁₋₆alkyl;
wherein with the proviso that R¹, R² and R³ are not H simultaneously;
or a pharmaceutically acceptable salt thereof.

9. A compound according to claim 8, wherein
R¹ is H, F, Cl or methoxy;
R² is H, Cl or methoxy;
R³ is H, ethyl, propyl, trifluoroethyl, methoxyethyl, methylthiazolyl, (methyl)₂thiazolyl, phenylthiazolyl or —C(X)—R⁷; wherein
X is O or S;
R⁷ is amino, trifluoropropyl, methylamino, dimethylamino, methylcarbonylamino, isopropylcarbonylamino, phenylcarbonylamino or tetrahydropyranyl;
R⁴ is H, methyl or ethyl;
R⁵ is H or methyl;
R⁶ is H or methyl;
wherein with the proviso that R¹, R² and R³ are not H simultaneously;
or a pharmaceutically acceptable salt thereof.

10. A compound according to claim 1, selected from selected from the group consisting of:
2-(3-chlorophenyl)benzotriazol-5-amine;
2-(4-chlorophenyl)benzotriazol-5-amine;
2-(4-methoxyphenyl)benzotriazol-5-amine;
2-(4-fluorophenyl)benzotriazol-5-amine;
2-(3-fluorophenyl)benzotriazol-5-amine;
2-(3-methoxyphenyl)benzotriazol-5-amine;
2-(4-methylsulfonylphenyl)benzotriazol-5-amine;
2-(4-ethylsulfonylphenyl)benzotriazol-5-amine;
2-[4-(2-methoxyethylsulfonyl)phenyl]benzotriazol-5-amine;
2-(4-isobutylsulfonylphenyl)benzotriazol-5-amine;
2-(4-chlorophenyl)-7-methyl-benzotriazol-5-amine;
2-(4-chlorophenyl)-N-methyl-benzotriazol-5-amine;
2-(4-chlorophenyl)-N-ethyl-benzotriazol-5-amine;
2-(4-chlorophenyl)-N-propyl-benzotriazol-5-amine;
2-(4-chlorophenyl)-N-(2-methoxyethyl)benzotriazol-5-amine;
N-cyclopentyl-2-phenyl-benzotriazol-5-amine;
N-(2-methoxyethyl)-2-(3-methoxyphenyl)benzotriazol-5-amine;
2-(3-chlorophenyl)-N-(2-methoxyethyl)benzotriazol-5-amine;
N-(2-methoxyethyl)-2-(4-methylsulfonylphenyl)benzotriazol-5-amine;
2-(4-fluorophenyl)-N-(tetrahydrofuran-3-ylmethyl)benzotriazol-5-amine;
2-(4-fluorophenyl)-N-tetrahydrofuran-3-yl-benzotriazol-5-amine;
2-(4-fluorophenyl)-N-(3-methoxypropyl)benzotriazol-5-amine;
2-(4-chlorophenyl)-N-(tetrahydrofuran-3-ylmethyl)benzotriazol-5-amine;
2-(4-chlorophenyl)-N-(cyclobutylmethyl)benzotriazol-5-amine;
2-(4-chlorophenyl)-N-(cyclopropylmethyl)benzotriazol-5-amine;
2-(4-chlorophenyl)-N-(cyclopentylmethyl)benzotriazol-5-amine;
2-(4-chlorophenyl)-N-(tetrahydrofuran-2-ylmethyl)benzotriazol-5-amine;
2-(4-chlorophenyl)-N-(2,2,2-trifluoroethyl)benzotriazol-5-amine;
2-(4-chlorophenyl)-N-cyclobutyl-benzotriazol-5-amine;
2-(4-chlorophenyl)-N-isopropyl-benzotriazol-5-amine;
2-(4-chlorophenyl)-N-cyclopentyl-benzotriazol-5-amine;
2-(4-chlorophenyl)-N-(2,2,2-trifluoro-1-methyl-ethyl)benzotriazol-5-amine;
2-(4-chlorophenyl)-N,N-dimethyl-benzotriazol-5-amine;
2-(4-chlorophenyl)-N,N-diethyl-benzotriazol-5-amine;
2-(4-chlorophenyl)-N-(2-methoxyethyl)-N-methyl-benzotriazol-5-amine;
2-(4-chlorophenyl)-N-ethyl-N-methyl-benzotriazol-5-amine;
3-[[2-(4-chlorophenyl)benzotriazol-5-yl]amino]-N-methyl-propanamide;
N-[2-(4-chlorophenyl)benzotriazol-5-yl]acetamide;
N-[2-(4-chlorophenyl)benzotriazol-5-yl]cyclohexanecarboxamide;
N-[2-(4-fluorophenyl)benzotriazol-5-yl]tetrahydrofuran-3-carboxamide;
N-[2-(4-fluorophenyl)benzotriazol-5-yl]tetrahydropyran-4-carboxamide;
N-[2-(4-fluorophenyl)benzotriazol-5-yl]tetrahydropyran-2-carboxamide;
N-[2-(4-fluorophenyl)benzotriazol-5-yl]cyclopentanecarboxamide;
3,3,3-trifluoro-N-[2-(4-fluorophenyl)benzotriazol-5-yl]propanamide;
N-[2-(4-chlorophenyl)benzotriazol-5-yl]tetrahydropyran-4-carboxamide;
N-[2-(4-chlorophenyl)benzotriazol-5-yl]tetrahydropyran-2-carboxamide;
N-[2-(4-chlorophenyl)benzotriazol-5-yl]-1,4-dioxane-2-carboxamide;
N-[2-(4-chlorophenyl)benzotriazol-5-yl]tetrahydrofuran-3-carboxamide;
N-[2-(4-chlorophenyl)benzotriazol-5-yl]cyclopentanecarboxamide;
N-[2-(4-chlorophenyl)benzotriazol-5-yl]tetrahydrofuran-2-carboxamide;
N-[2-(4-chlorophenyl)benzotriazol-5-yl]-3,3,3-trifluoro-propanamide;
N-[2-(4-chlorophenyl)benzotriazol-5-yl]-4,4,4-trifluoro-butanamide;
[2-(4-chlorophenyl)benzotriazol-5-yl]thiourea;
1-[2-(4-chlorophenyl)benzotriazol-5-yl]-3-methyl-thiourea;
N-[[2-(4-chlorophenyl)benzotriazol-5-yl]carbamothioyl]acetamide;
N-[[2-(4-chlorophenyl)benzotriazol-5-yl]carbamothioyl]cyclopentanecarboxamide;
N-[[2-(4-chlorophenyl)benzotriazol-5-yl]carbamothioyl]-2-methyl-propanamide;
N-[[2-(4-chlorophenyl)benzotriazol-5-yl]carbamothioyl]benzamide;
N-[[2-(4-chlorophenyl)-6-methyl-benzotriazol-5-yl]carbamothioyl]benzamide;
[2-(4-chlorophenyl)-6-methyl-benzotriazol-5-yl]thiourea;
3-[2-(4-chlorophenyl)benzotriazol-5-yl]-1,1-dimethyl-thiourea;
N-[2-(4-chlorophenyl)benzotriazol-5-yl]-4-isopropyl-thiazol-2-amine;
N-[2-(4-chlorophenyl)benzotriazol-5-yl]-4-phenyl-thiazol-2-amine;
N-[2-(4-chlorophenyl)benzotriazol-5-yl]-4-methyl-thiazol-2-amine; and,
N-[2-(4-chlorophenyl)benzotriazol-5-yl]-4,5-dimethyl-thiazol-2-amine;
or a pharmaceutically acceptable salt thereof.

11. A compound according to claim 1, selected from
2-(3-chlorophenyl)benzotriazol-5-amine;
2-(4-methoxyphenyl)benzotriazol-5-amine;
2-(3-methoxyphenyl)benzotriazol-5-amine;
2-(4-chlorophenyl)-7-methyl-benzotriazol-5-amine;
2-(4-chlorophenyl)-N-ethyl-benzotriazol-5-amine;
2-(4-chlorophenyl)-N-propyl-benzotriazol-5-amine;
2-(4-chlorophenyl)-N-(2,2,2-trifluoroethyl)benzotriazol-5-amine;
2-(4-chlorophenyl)-N,N-diethyl-benzotriazol-5-amine;
2-(4-chlorophenyl)-N-(2-methoxyethyl)-N-methyl-benzotriazol-5-amine;
N-[2-(4-fluorophenyl)benzotriazol-5-yl]tetrahydropyran-4-carboxamide;
N-[2-(4-chlorophenyl)benzotriazol-5-yl]tetrahydropyran-2-carboxamide;
N-[2-(4-chlorophenyl)benzotriazol-5-yl]-4,4,4-trifluoro-butanamide;
1-[2-(4-chlorophenyl)benzotriazol-5-yl]-3-methyl-thiourea;
N-[[2-(4-chlorophenyl)benzotriazol-5-yl]carbamothioyl]acetamide;
N-[[2-(4-chlorophenyl)benzotriazol-5-yl]carbamothioyl]-2-methyl-propanamide;
N-[[2-(4-chlorophenyl)benzotriazol-5-yl]carbamothioyl]benzamide;
N-[[2-(4-chlorophenyl)-6-methyl-benzotriazol-5-yl]carbamothioyl]benzamide;
[2-(4-chlorophenyl)-6-methyl-benzotriazol-5-yl]thiourea;
3-[2-(4-chlorophenyl)benzotriazol-5-yl]-1,1-dimethyl-thiourea;
N-[2-(4-chlorophenyl)benzotriazol-5-yl]-4-phenyl-thiazol-2-amine;
N-[2-(4-chlorophenyl)benzotriazol-5-yl]-4-methyl-thiazol-2-amine; and
N-[2-(4-chlorophenyl)benzotriazol-5-yl]-4,5-dimethyl-thiazol-2-amine;
or a pharmaceutically acceptable salt thereof.

12. A process for the preparation of a compound according claim 1 comprising at least one of the following steps,
(a) coupling and cyclization of a diazonium salt (III), 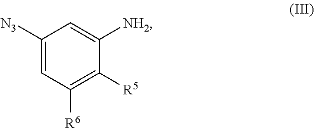
with a compound of formula (IV), 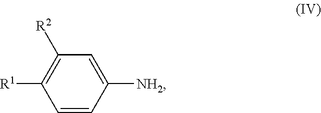
in the presence of copper (II) sulfate and ammonium hydroxide;
(b) coupling of a compound of formula (I-1), 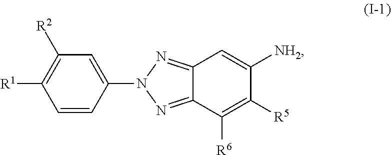
with a compound of formula (V), R³-L¹ (V), in the presence of a base or a Lewis acid;
(c) coupling of a compound of formula (I-2), 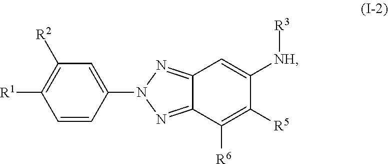
with a compound of formula (VI), R⁴-L² (VI), in the presence of a base;
wherein L¹ is N═C═S, halogen or OH; L² is halogen.

13. A compound according to claim 1 for use as therapeutically active substance.

14. A pharmaceutical composition comprising a compound in accordance with a claim 1 and a therapeutically inert carrier.

15. The use of a compound according to claim 1 for the treatment of HBV infection.

16. The use of a compound according to claim 1 for the preparation of a medicament for the treatment of HBV infection.

17. The use of a compound according to claim 1 for the inhibition of HBeAg.

18. The use of a compound according to claim 1 for the inhibition of HBsAg.

19. The use of a compound according to claim 1 for the inhibition of HBV DNA.

20. A compound according to claim 1 for use in the treatment or prophylaxis of HBV infection.

21. A compound according to claim 1, when manufactured according to a process of claim 12.

22. A method for the treatment of HBV infection, which method comprises administering an effective amount of a compound according to claim 1.
